SEQUENCE LISTING (10 sequences)

SEQ ID NO: 1 - protein, 7 aa, synthetic construct
TWLPLPR

SEQ ID NO: 2 - protein, 8 aa, synthetic construct
YVLLPSPK

SEQ ID NO: 3 - protein, 7 aa, synthetic construct
KVPPLLY

SEQ ID NO: 4-7 - skipped (4 entries)

SEQ ID NO: 8 - protein, 7 aa, synthetic construct
TWLPLPR

SEQ ID NO: 9 - protein, 8 aa, synthetic construct
YVLLPSPK

SEQ ID NO: 10 - protein, 7 aa, synthetic construct
KVPPLLY

(19) United States
(12) Patent Application Publication
(10) Pub. No.: US 2025/0134794 A1
(43) Pub. Date: May 1, 2025

(54) BIOACTIVE WALNUT PEPTIDES AND COMPOSITIONS FOR TOPICAL TREATMENT OF SKIN

(71) Applicant: L'OREAL, Paris (FR)

(72) Inventors: Rebecca BARRESI, Robbinsville, NJ (US); I-chien LIAO, Princeton, NJ (US); Charbel BOUEZ, Hoboken, NJ (US)

(73) Assignee: L'OREAL, Paris (FR)

(21) Appl. No.: 18/932,836

(22) Filed: Oct. 31, 2024

Related U.S. Application Data
(60) Provisional application No. 63/594,586, filed on Oct. 31, 2023.

(30) Foreign Application Priority Data
Feb. 27, 2024 (FR) 2401882

(51) Int. Cl. A61K 8/64; A61K 9/00; A61K 38/08; A61Q 19/02

(52) U.S. Cl. CPC A61K 8/645; A61K 9/0014; A61K 38/08; A61Q 19/02

(57) ABSTRACT

Bioactive walnut peptides and use of the bioactive walnut peptides for topical application to the skin are described. The bioactive walnut peptides, which may be incorporated into a pharmaceutical or cosmetic compositions, are useful for treating, preventing, and improving damage to skin and combating or preventing physiological changes associated with skin aging.

### CROSS-REFERENCE TO RELATED APPLICATIONS

This application claims benefit of U.S. Ser. No. 63/594,586, filed Oct. 31, 2023, and benefit of French Application No. FR 2401882, filed on Feb. 27, 2024, which are incorporated herein by reference in their entirety.

### REFERENCE TO THE SEQUENCE LISTING

The Sequence Listing submitted Oct. 31, 2024 as an XML file named “Sequence Listing,” created on Oct. 30, 2024 and having a size of 11 kilobytes is hereby incorporated by reference pursuant to 37 C.F.R. § 1.52(e)(5).

### FIELD OF THE DISCLOSURE

The present disclosure relates to bioactive walnut peptides and use of the walnut peptides for topical application to the skin. The walnut peptides and compositions comprising them are useful for improving the appearance of skin, preventing, or reversing skin aging, and treating various skin conditions associated with aging.

### BACKGROUND

Skin is a unique and complex organ extending over the entire body. It is critical for protecting the body from external threats, controlling body temperature, and preventing loss of moisture. It also serves as a sensory organ and produces vitamin D. There are different types of skin at different locations of the body. For example, facial skin is different from that of the scalp, and even the skin on the palm of the hand is different than skin on the back of the hand.

Skin makes up the integumentary system, which includes three layers: the epidermis, the dermis, and the hypodermis. The epidermis is the outermost layer of skin and the first line of defense from external threats such as microbes. The human epidermis is principally composed of keratinocytes contains other types of cells including the melanocytes and the Langerhans' cells. Each of these cell types contribute, through their specific function, to the essential role played by the skin. Melanocytes provide color and tone characteristic of the skin. The epidermis is essentially waterproof and therefore helps maintain hydration and prevents the body from absorbing water when bathing. Unlike other cells, epidermal cells have a unique ability to regenerate and therefore can heal after being wounded.

The middle layer of skin that lies right below the epidermis, is the dermis, which includes connective tissue, collage, elastin, hair follicles lymph nodes, and sweat glands. The dermis provides a solid support for the epidermis. It is also its feeder layer. The dermis includes mainly of fibroblasts, but leukocytes, mast cells or tissue macrophages are also present. The dermis also contains blood vessels and nerve fibers. The acellular part (i.e., the area in between the cells) of the dermis is called extracellular matrix. The extracellular matrix of skin is composed of various extracellular components including proteins, in particular collagen fibers and elastin. Other extracellular matrix components of skin include glycosaminoglycans (e.g., hyaluronic acid, chondroitin sulfate, dermatan sulfate, keratan sulfate, heparan sulfate, etc.), proteoglycans (e.g., fibromodulin, decorin, biglycan, perlecan, heparan sulfate proteoglycan 2, agrin, versican, aggrecan, lumican, collagen type IX, collagen type XII, collagen type XIV, testican 1, testican 2, etc.) and various glycoproteins (e.g., fibrillin 1, thrombospondin-1 and -2, tenascin-C and -X, osteopontin, fibronectin, laminin-5 and -6, vitronectin, etc.). These extracellular components are synthesized by dermal fibroblasts, which make dermal fibroblasts the primary constituent in the structural assembly of the dermis.

The extracellular matrix is a highly heterogeneous amalgam of morphologically diverse architectural entities. It organizes and imparts structural integrity to individual tissues, in addition to modulating cell behavior by interacting with cell surface receptors and soluble growth factors. Dysfunctions and changes in components of the extracellular matrix can therefore interfere with both tissue integrity and cell performance. Dysfunctions and changes in components of the extracellular matrix of skin and mucosa in humans can lead to skin aging, skin atrophy, damaged skin, wounded skin, and other problems.

The hypodermis is the deepest layer of the skin and is mostly fat and connective tissue. It connects the skin to underlying fascia (tissue) of the bones and muscles. The hypodermis not always classified as an official layer of skin, but it nonetheless provides important functions like storing energy, insulating the body, and preventing the skin from sagging. The hypodermis houses fat-storing cells that function as energy reservoirs and act as a cushion against impact.

The skin ages over time and wrinkles, dark spots, and loss of elasticity and volume can ensue. Skin aging includes both chronological aging (endogenous aging), which is a natural aging that occurs over time due to natural biological processes, and degenerative aging due to outside influences such as exposure to damaging sunlight, pollution, lack of hydration, etc. Ultraviolet rays such as UVB having a wavelength in the range of 280 nm to 320 nm and are known to cause skin damage and accelerate aging. UVB rays stimulate reactive oxygen species (ROS) and free radicals in skin cells, which speed the intracellular signaling system inducing oxidative stress on biomolecules such as DNA, proteins, and lipids. Increasing the oxidative stress of skin cells may cause stimulation of keratinocytes of the epidermis or fibroblasts of the dermis, and through a series of intracellular signal transduction, the expression of genes such as matrix metalloproteinase (MMP), a collagen degrading enzyme, may be increased.

There are many treatments available for treating skin that promote anti-aging benefits. For example, the importance of collagen in the aging process has led to the development of many collagen-containing topical products. Other components such as retinoic acid, vitamin C and hyaluronic acid are used in topical formulations to help stimulate collagen synthesis. Some cosmetic procedures, such as laser skin resurfacing, are used for reducing facial wrinkles and skin irregularities. The topical administration of proteins known to inhibit one or more signaling pathways that exhibit altered activity after UV exposure have been explored. However, there are difficulties associated with the use of whole proteins, which cannot be effectively delivered though the epidermis. Also, large proteins can be unstable, difficult to formulate, and not topically effective. Despite the number of product and treatments that have been proposed none of them satisfactory evoke the intrinsic repair and renewal mechanisms of the skin.

### SUMMARY OF THE DISCLOSURE

The instant disclosure relates to bioactive walnut peptides and use of the walnut peptides for topical application to the skin. The bioactive walnut peptides, which may be incorporated into a pharmaceutical or cosmetic compositions, treat and prevent conditions that negatively impact skin aging. Moreover, topical application of the bioactive walnut peptides to the skin surprisingly reduces age-related inflammation, increases epidermal growth factor (EGF), hepatocyte growth factor (HGF), and transforming growth factor alpha (TGFa), promotes expression of epidermal stem cells, induces autophagy, and improves mitochondrial function. It was surprising to find that bioactive walnut peptides influence multiple physiological pathways that benefit skin. Due to the plethora of benefits, the bioactive walnut peptides and pharmaceutical and cosmetic composition containing them are particularly suitable for preventing and treating skin aging and improving or reversing the effects of skin aging.

Walnuts are one of the most widely distributed and oldest nuts in the world. They have high nutritional value and are rich in oleic acid, linoleic acid, α-linolenic acid, and other unsaturated fatty acids, vitamins, and proteins. Walnuts are commonly used to make walnut oil because they contain a high lipid content. The residue remaining after lipid extraction is considered a by-product although it contains walnut protein and other useful components. Walnut protein is mainly composed of albumin, globulin, gliadin and glutenin.

Bioactive walnut peptides can be derived from walnut proteins or can be synthesized. For example, walnut peptides are obtained via enzymatic hydrolysis, fermentation hydrolysis, or chemical hydrolysis of walnut proteins or synthetically produced, for example, by solid-phase synthesis. Bioactive peptides that are particularly useful according to the instant disclosure typically have a molecular weight less than 6,000 Da and often less than 1,000 Da. Such walnut peptides can have from 2 to 50 amino acid residues but typically have from 2 or 3 amino acid residues up to about 20 amino acid residues.

Preferably, the bioactive walnut peptides have a minimum of 2 or 3 amino acid residues up to about 20 amino acid residues and impart a positive physiological or dermatological effect on skin cells. Bioactive peptides include amino acids joined by covalent bonds, also referred to as amide or peptide bonds, whereas proteins are polypeptides with a greater molecular weight (MW), i.e., having more than 50 amino acid residues. Bioactive walnut peptides usually display hormone or drug-like activities and are classified based on their mode of action. Many bioactive peptides share some structural features for example, an amino acid residue length for 2 to 20 amino acids.

Useful bioactive walnut peptides according to the instant disclosure include often include one or more amino acid residues selected from leucine, proline, or combinations thereof. In further embodiments, the walnut peptides include three or more amino acid residues selected from leucine, proline, or combinations thereof. Nonlimiting examples of amino acid residues within the walnut peptides that include leucine and proline include Leu-Pro-Leu (LPP), Leu-Leu-Pro, Pro-Pro-Leu (PPL), and Pro-Leu-Pro (PLP). Further nonlimiting examples of amino acid sequence that can be included in the bioactive walnut peptides include Thr-Trp-Leu-Pro-Leu-Pro-Arg (TWLPLPR), Tyr-Val-Leu-Leu-Pro-Ser-Pro-Lys (YVLLPSPK), Lys-Val-Pro-Pro-Leu-Leu-Tyr (KVPPLLY), and combinations thereof.

In various embodiments, one or more of the following walnut peptides are preferred:

Peptide I
Thr-Trp-Leu-Pro-Leu-Pro-Arg (TWLPLPR),
Peptide II
Tyr-Val-Leu-Leu-Pro-Ser-Pro-Lys (YVLLPSPK),
and/or
Peptide III
Lys-Val-Pro-Pro-Leu-Leu-Tyr (KVPPLLY).

As already mentioned, the bioactive walnut peptides are particularly useful for topical application to the skin. Accordingly, the instant disclosure is drawn to the use of walnut peptides in methods for treating the skin. In various embodiments, the one or more walnut peptides are applied to the skin in a pharmaceutical or cosmetic composition, which typically includes a physiologically acceptable carrier, for example, water and optionally water-soluble solvents. The pharmaceutical or cosmetic composition includes an amount of the one or more bioactive walnut peptides sufficient to ensure a therapeutically effective amount of the one or more walnut peptides is administered to the skin during use.

The bioactive walnut peptides are useful for treating age-related inflammation. For example, the bioactive walnut peptides surprisingly and advantageously reduce, treat, or prevent pro-inflammatory cytokines in the skin. Proinflammatory cytokines are among the first factors to be produced in response to damage to the skin, and they regulate the functions of immune cells in epithelialization. In various embodiments, the bioactive peptides of the instant disclosure prevent or down-regulate production, release, or abundance of Interleukin-6 (IL-6), Interleukin-8 (IL-8), or a combination thereof.

The bioactive walnut peptides are useful for stimulating skin cells called fibroblasts, which produce collagen and elastin to clarify, thicken, and tighten skin. Growth factors are immensely important for the regulation of cell processes in the human body, including the skin. The bioactive walnut peptides of the instant disclosure are useful potentiating production, release, and abundance of epidermal growth factor (EGF). EGF is a reparative compound that signals to cells to boost collagen and elastin production. This helps with cellular repair and renewal, resulting in a more radiant, youthful complexion. In addition, the bioactive walnut peptides are useful for potentiating production, release, and abundance of hepatocyte growth factor (HGF). Increasing HGF improves tissue fibrosis and reverse the imbalance of collagen metabolism. Finally, the bioactive walnut peptides are useful for potentiating production, release, and abundance of transforming growth factor alpha (TGFa or TGF-α). TGFa is a transforming growth factor that functions as a ligand for the epidermal growth factor receptor, which activates a signaling pathway for cell proliferation, differentiation, and development. Therefore, increasing TGFa can expedite rejuvenation and repair of the skin.

In further embodiments, the bioactive walnut peptides of the instant disclosure are useful for potentiating, inducing, and increasing autophagy in skin, for example, in senescent fibroblasts. Senescent cells are characterized by their inability to proliferate. They tend to accumulate with age and contribute to age-related skin changes and pathologies. Inducing autophagy allows the body to destroy and metabolize damaged or redundant cellular components occurring in vacuoles within cells. A natural reduction of autophagy occurs with age. Thus, restoration or inducement of autophagy helps to improve skin health and prevent premature skin aging, for example, skin aging due to sun damage.

Due to their bioactive properties, including those described above, the bioactive walnut peptides are useful in methods for improving the appearance of skin; reducing treating, or preventing age-related inflammation of the skin and skin conditions associated with age-related inflammation; promoting migration of fibroblasts; potentiating hyaluronic acid synthesis, increasing or promoting synthesis or abundance of collagen, elastin, and/or fibronectin; improving elasticity of skin; promoting autophagy in the skin; and combinations thereof.

In further embodiments, the bioactive walnut peptides are useful in one or more methods for depigmenting the skin, lightening the skin, brightening the skin, treating hyperpigmentation, and treating melasmic skin. Similarly, the bioactive walnut peptides are useful in methods for evening out skin tone.

In various embodiments, use of two or more bioactive walnut peptides is preferred. The two or more bioactive walnut peptides may have similar activities or may provide different activities that benefit the skin. In further embodiments, use of three or more bioactive walnut peptides is preferred. The use of multiple bioactive peptides allows for the modification of more than one physiological mechanism in the treatment of skin. For example, one or more bioactive walnut peptide may be useful for preventing and/or treating age-related inflammation while another bioactive walnut peptide may be useful for stimulating fibroblasts that produce collagen and elastin to clarify, thicken, and tighten skin. Moreover, combinations of bioactive walnut peptides can interact synergistically and benefits beyond the sum of the peptides' individual contributions. For example, the synergistic activity of a combination can be at least 5%, at least 10%, or at least 25% greater than the sum of the individual activities of the corresponding amounts of the bioactive walnut peptides.

The one or more bioactive walnut peptides are often incorporated into a pharmaceutical or cosmetic composition for application to the skin. Pharmaceutical and cosmetic composition typically include one or more bioactive walnut peptides and one or more physiologically acceptable carrier, for example water. Nonlimiting examples of physiologically acceptable carriers include water, water soluble solvents such as alcohols, polyols, and glycols, fatty compound such as oils, triglycerides, fatty acids, fatty alcohols, and the like. Pharmaceutical and cosmetic compositions include lotions, creams, serums, sprays, emulsions, gels, powders, dispersions, ointments, sticks, pastes, and foams.

### BRIEF DESCRIPTION OF THE DRAWINGS

Implementation of the present technology is described, by way of example only, with reference to the attached figures, wherein:

FIG. 1(a) shows the concentration of Interleukin-6 (IL-6) released from Adult Dermal Human Fibroblasts in response to treatment with 100 uM and 250 uM Peptide I, Peptide II, and Peptide III.

FIG. 1(b) shows the concentration of Interleukin-8 (IL-8) released from Adult Dermal Human Fibroblasts in response to treatment with 100 uM and 250 uM Peptide I, Peptide II, and Peptide III.

FIG. 2(a) shows the amounts of epidermal growth factor (EGF) generated with reconstructed human epidermis in response to treatment with 250 uM Peptide I, Peptide II, and Peptide III, and combinations thereof.

FIG. 2(b) shows the amounts of hepatocyte growth factor (HGF) generated by reconstructed human epidermis in response to treatment with 250 uM Peptide I, Peptide II, and Peptide III, and combinations thereof.

FIG. 2(c) shows the amounts of transforming growth factor alpha (TGFa) generated by reconstructed human epidermis in response to treatment with 250 uM Peptide I, Peptide II, and Peptide III, and combinations thereof.

FIG. 4 shows the mean intensity of active mitochondria of Adult Dermal Human Fibroblasts in response to treatment with 250 uM of Peptide I, Peptide II, Peptide III, and combinations thereof.

The various aspects of the disclosure are not limited to the results, arrangements, and representations shown in the drawings.

### DETAILED DESCRIPTION OF THE DISCLOSURE

The instant disclosure is drawn to bioactive walnut peptides and their topical use for treating skin. Bioactive walnut peptides include two to several dozen amino acids connected to one another through peptide bonds. Their molecular weight is generally less than 6000 Da, preferably less than 3,000 Da, and more preferably less than 1,000 Da. The term “peptide” in accordance with the present disclosure is a compound that includes an uninterrupted sequence of at least two amino acids within its structure and has a maximum of about 50 amino acid. The terms “di-peptide” or “dipeptide” as used herein refer to a compound that includes an uninterrupted sequence of two amino acids within its structure. The terms “tri-peptide” or “tripeptide” as used herein refer to a compound that includes an uninterrupted sequence of three amino acids within its structure. As used herein, a “tetra-peptide” or “tetrapeptide” is a compound that includes an uninterrupted sequence of four amino acids within its structure. These amino acids are indicated herein using a traditional one letter convention from left (N-terminal end) to right (C-terminal end). In this nomenclature, G is glycine, H is histidine, K is lysine, E is glutamic acid, and the like, according to well known and accepted nomenclature in the art.

A “bioactive” peptide for purposes of the instant disclosure has a minimum of 2 or 3 amino acid residues up to about 20 amino acid residues in length and has a measurable physiological effect on skin cells. Bioactive peptides include amino acids joined by covalent bonds, also referred to as amide or peptide bonds, whereas proteins are polypeptides with a greater molecular weight (MW) and typically more than 50 amino acid residues. Bioactive peptides usually display hormone or drug-like activities and are classified based on their mode of action. Many bioactive peptides share some structural features that include, for example, a peptide residue length of 2 to 20 amino acids.

The term “amino acid” as used herein includes and encompasses all naturally occurring amino acids, either in the D- or L-configuration if optically active, and the known non-native, synthetic, and modified amino acids, such as homocysteine, ornithine, norleucine, and p-valine. A list of non-natural amino acids may be found in The Peptides, Vol. 5 (1983), Academic Press, Chapter VI, by D. C. Roberts and F. Vellaccio, which is incorporated herein by reference in its entirety. The amino acids in the peptides of the present invention may be present in their natural L-configuration, unnatural D-configuration, or as a racemic mixture.

As used herein, the term “peptide” shall also refer to salts, deprotected forms, acylated forms of the peptide, deacylated forms of the peptide, enantiomers, diastereomers, racemates, prodrugs and hydrates of the above-mentioned peptide. Diastereomers of the peptide are obtained when the stereochemical or chiral center of one or more amino acids is changed. The enantiomer has the opposite stereochemistry at all chiral centers. In various embodiments, the C-terminal of a peptide is synthesized as an amide to neutralize negative charge created by the C-terminal COOH. This modification can be added to help prevent enzyme degradation.

The term “prodrug” refers to any precursor compound which can generate or to release the above-mentioned peptide under physiological conditions. Such prodrugs are for instance larger peptides which are selectively cleaved to form the peptide of the invention. Further prodrugs are protected amino acids having protecting groups at the carboxylic acid and/or amino group. Suitable protecting groups for amino groups include, for example, the benzyloxycarbonyl, t-butyloxycarbonyl (BOC), formyl, and acetyl or acyl group. Suitable protecting groups for the carboxylic acid group are esters such as benzyl esters or t-butyl esters.

The instant disclosure includes methods for treating skin comprising topical application of a therapeutically effective amount of one or more walnut peptides to the skin. In various embodiments, the one or more bioactive walnut peptides have a molecular weight of less than 10,000 Da. In further embodiments, the one or more bioactive walnut peptides have a molecular weight of less than 8,000 Da, less than 6,000 Da, less than 5,000 Da, less than 4,000 Da, less than 3,000 Da, less than 2,000 Da, less than 1,000 Da, and even less than 500 Da. Preferably, the one or more bioactive walnut peptides have a molecular weight of less than 5,000 Da, more preferably less than 2,000 Da, and even more preferably less than 1,000 Da.

The number of amino acid residues in the one or more bioactive walnut peptides will vary and can be limited based on the molecular weights described above. Nonetheless, in various embodiments, the one or more bioactive walnut peptides comprise from 2 to about 50 amino acid residues. In further embodiments, the one or more bioactive peptides have from 2 to about 25 amino acid residues, from about 2 to about 20 amino acid residues, about 2 to about 18 amino acid residues, about 2 to about 15 amino acid residues, about 2 to about 12 amino acid residues, about 2 to about 10 amino acid residues, about 3 to about 25 amino acid residues, about 3 to about 20 amino acid residues, about 3 to about 18 amino acid residues, about 3 to about 18 amino acid residues, about 3 to about 15 amino acid residues, about 3 to about 12 amino acid residues, or about 3 to about 10 amino acid residues. Preferably, the one or more bioactive peptides comprise 2 to 20 amino acid residues, more preferably 2 to about 15 amino acid residues, even more preferably 3 to about 12 amino acid residues.

In various embodiments, the one or more bioactive walnut peptides include one or more amino acid residues selected from leucine, proline, and combinations thereof. In a further embodiments, the one or more bioactive walnut peptides include three or more amino acid residues selected from leucine, proline, or combination thereof. Nonlimiting examples of bioactive of three or more amino acid residues selected from leucine, proline, and combinations thereof include Leu-Pro-Leu, Leu-Leu-Pro, Pro-Pro-Leu, and Pro-Leu-Pro. For example, the one or more bioactive walnut peptides may comprise an amino acid sequence selected from Thr-Trp-Leu-Pro-Leu-Pro-Arg, Tyr-Val-Leu-Leu-Pro-Ser-Pro-Lys, or Lys-Val-Pro-Pro-Leu-Leu-Tyr. As already mentioned, the bioactive walnut peptides include salts, deprotected forms, acylated forms, deacylated forms, enantiomers, diastereomers, racemates, prodrugs, and hydrates of a particular amino acid sequence.

In a preferred embodiment at least one, and preferably all, of the one or more bioactive walnut peptides are selected from Peptide I, Peptide II, and Peptide III including salts, deprotected forms, acylated forms, deacylated forms, enantiomers, diastereomers, racemates, prodrugs, and hydrates thereof.

Peptide I
TWLPLPR (Thr-Trp-Leu-Pro-Leu-Pro-Arg),
Peptide II
YVLLPSPK (Tyr-Val-Leu-Leu-Pro-Ser-Pro-Lys),
and
Peptide III
KVPPLLY (Lys-Val-Pro-Pro-Leu-Leu-Tyr).

In various embodiments, use of two or more bioactive walnut peptides is desirable. The two or more bioactive walnut peptides may share similar activities or may provide different activities that benefit the skin. In further embodiments, use of three or more bioactive walnut peptides is preferred. The use of multiple bioactive peptides allows for reliance on more than one physiological mechanism for the treatment of skin. For example, one or more bioactive walnut peptide may be useful for preventing and/or treating age-related inflammation while another bioactive walnut peptide may be useful for stimulating fibroblasts that produce collagen and elastin to clarify, thicken, and tighten skin. Moreover, as shown later, combinations of bioactive walnut peptides interact synergistically to provide benefits beyond the sum of the peptides' individual contributions. For example, the synergistic activity of a combination of bioactive walnut peptides can be at least 5%, preferably at least 10%, more preferably at least 25% greater than the sum of the activity of the corresponding individual amounts of the bioactive walnut peptides.

Combinations of Peptide I, Peptide II, and Peptide III are particularly useful. For example, a combination of Peptide I and Peptide II, a combination of Peptide I and Peptide III, a combination of Peptide II and Peptide III, or a combination of Peptide I, Peptide II, and Peptide III can be used. The peptides in the combinations can be included in various weight ratios to one another as described below.

Peptide I and Peptide II may be used together in a weight ratio of about 1:10 to about 10:1. In further embodiments, Peptide I and Peptide II may be used together in a weight ratio of about 8:1 to about 1:8, about 5:1 to about 1:5, about 4:1 to about 1:4, about 3:1 to about 1:3, about 2:1 to about 1:2, or about 1:1.

Peptide I and Peptide III may be used together in a weight ratio of about 1:10 to about 10:1. In further embodiments, Peptide I and Peptide III may be used together in a weight ratio of about 8:1 to about 1:8, about 5:1 to about 1:5, about 4:1 to about 1:4, about 3:1 to about 1:3, about 2:1 to about 1:2, or about 1:1.

Peptide II and Peptide III may be used together in a weight ratio of about 1:10 to about 10:1. In further embodiments, Peptide II and Peptide III may be used together in a weight ratio of about 8:1 to about 1:8, about 5:1 to about 1:5, about 4:1 to about 1:4, about 3:1 to about 1:3, about 2:1 to about 1:2, or about 1:1.

Bioactive walnut peptides are useful for treating age-related inflammation and for preventing, reducing, and even reversing the effects of skin aging. The bioactive walnut peptides advantageously reduce, treat, or prevent pro-inflammatory cytokines in the skin. Proinflammatory cytokines are among the first factors to be produced in response to skin damage, and they regulate the functions of immune cells in epithelialization. In various embodiments, the bioactive peptides of the instant disclosure prevent production, release, or abundance of Interleukin-6 (IL-6), Interleukin-8 (IL-8), or a combination thereof.

The bioactive walnut peptides are useful for stimulating skin cells called fibroblasts, which produce collagen and elastin to clarify, thicken, and tighten skin. Growth factors are immensely important for the regulation of cell processes in the human body, including the skin. In particular, the bioactive walnut peptides of the instant disclosure are useful potentiating production, release, and abundance of epidermal growth factor (EGF). EGF is a reparative compound that signals to cells to boost collagen and elastin production. This helps with cellular repair and renewal, resulting in a more radiant, youthful complexion. In addition, the bioactive walnut peptides are also useful for potentiating production, release, and abundance of hepatocyte growth factor (HGF). Increasing HGF improves tissue fibrosis and reverse the imbalance of collagen metabolism. Finally, the bioactive walnut peptides are useful for potentiating production, release, and abundance of transforming growth factor alpha (TGFa or TGF-α). TGFa is a transforming growth factor that functions as a ligand for the epidermal growth factor receptor, which activates a signaling pathway for cell proliferation, differentiation, and development. Therefore, increasing TGFa can expedite rejuvenation and repair of the skin.

In further embodiments, the bioactive walnut peptides of the instant disclosure are useful for potentiating, inducing, or increasing autophagy, for example, in senescent fibroblasts. Senescent cells are characterized by their inability to proliferate. They tend to accumulate with age and contribute to age-related skin changes and pathologies. Inducing autophagy allows the body to destroy and metabolize damaged or redundant cellular components occurring in vacuoles within cells. A natural reduction of autophagy occurs with age. Thus, restoration of autophagy using the bioactive peptides of the instant disclosure contributes to improving or preventing premature skin aging, for example, skin aging due to sun damage.

Due to their bioactive properties, including those described above, the bioactive walnut peptides are useful in methods for improving the appearance of skin; reducing, treating, and/or preventing skin aging and/or the effects of skin aging; increasing fibroblast proliferation; stimulating epithelial cell proliferation, motility, morphogenesis, and/or angiogenesis; promoting migration of fibroblasts; increasing abundance or synthesis of hyaluronic acid, and/or increasing synthesis or abundance of collagen, elastin, and/or fibronectin; improving elasticity of skin; and/or improving autophagy in skin fibroblasts.

The one or more bioactive walnut peptides are often incorporated into a pharmaceutical or cosmetic composition for application to the skin. Pharmaceutical and cosmetic compositions typically include one or more bioactive walnut peptides and a physiologically acceptable carrier. A “physiological carrier” as used herein is a carrier that is appropriate and safe for application to the skin of a human. A particularly common physiologically acceptable carriers is water. However, physiologically acceptable carriers can be oil, fats, organic solvents, and the like, provided they are appropriate and safe for application to the skin. Nonlimiting examples of physiologically acceptable carriers include water, water-soluble solvents such as alcohols, polyols, and glycols, fatty compound such as oils, triglycerides, fatty acids, fatty alcohols, and the like. The pharmaceutical and cosmetic compositions of the instant disclosure can be lotions, creams, serums, sprays, emulsions, gels, powders, dispersions, ointments, sticks, pastes, or foams.

In a preferred embodiment, the one or more bioactive walnut peptides reduce, treat, or prevent production, release, or abundance of pro-inflammatory cytokines in the skin, for example, Interleukin-6 (IL-6), Interleukin-8 (IL-8), or a combination thereof.

In a preferred embodiment, the one or more bioactive walnut peptides potentiate production, release, or abundance of epidermal growth factor (EGF), hepatocyte growth factor (HGF), and/or transforming growth factor alpha (TGFa) in the skin.

In a preferred embodiment, the one or more bioactive walnut peptides potentiate or upregulate autophagy in skin, for example in aged or senescent fibroblasts.

In a preferred embodiment, the one or more bioactive walnut peptides potentiate or improve mitochondrial function or activity of skin cells, for example, in fibroblasts or in senescent fibroblasts.

Bioactive walnut peptides can be derived by hydrolysis of walnut proteins into small molecular peptides with molecular weights between the molecular weight of individual amino acids and the molecular weights of the proteins. This can be carried out using biological or chemical methods. For example, bioactive peptides can be prepared by enzymatic procedures, fermentation, and with chemical methods.

Bioactive walnut peptides can be synthesized by coupling the carboxyl group or C-terminus of one amino acid to the amino group or N-terminus of another. Due to the possibility of unintended reactions, protecting groups are sometimes necessary. Chemical peptide synthesis starts at the C-terminal end of the peptide and ends at the N-terminus. Peptides can be synthesized either by solid-phase peptide synthesis, by liquid-phase peptide synthesis, or by fragment condensation. In principle, the seemingly simple formation of a peptide bond can be accomplished using all the procedures available in organic chemistry for the synthesis of carboxylic acid amides.

The general process for synthesizing peptides on solid-phase (e.g., resin) starts by attaching the first amino acid, the C-terminal residue, to the resin. To prevent the polymerization of the amino acid, the alpha amino group and the reactive side chains are protected with a temporary protecting group. Once the amino acid is attached to the resin, the resin is filtered and washed to remove byproducts and excess reagents. Next, the N-alpha protecting group is removed in a deprotection process, and the resin is again washed to remove byproducts and excess reagents. Then the next amino acid is coupled to the attached amino acid. This is followed by another washing procedure, which leaves the resin-peptide ready for the next coupling cycle. The cycle is repeated until the peptide sequence is complete. Then typically, all the protecting groups are removed, and the peptide resin is washed, and the peptide is cleaved from the resin.

Enzymatic hydrolysis involves using commercial enzymes to obtain bioactive peptides. The enzymes are responsible for cleaving the peptide bonds established in the protein, thereby releasing the encrypted peptide. For the enzyme to carry out its activities, it is important for the enzyme bind the substrate and continue with enzymatic catalysis. The enzyme has specific active sites containing residues that form temporary bonds with the substrate and residues that catalyze the reaction with the substrate. In this way, binding sites and catalytic sites are formed, respectively. The bonds forming the enzyme-substrate complex are usually hydrogen bonds, hydrophobic bonds, or Van der Waals interactions. Enzymatic hydrolysis can generally be carried out in three ways: (i) under traditional batch conditions; (ii) using immobilized enzymes; or (iii) using ultrafiltration membranes. Numerous proteolytic enzyme are known and include those described, for example, in Cruz-Casas et al., Enzymatic Hydrolysis and Microbial Fermentation: The Most Favorable Biotechnological Methods for the Release of Bioactive Peptides (FOOD CHEM (OXF). 3:100047, Dec. 30, 2021), which is incorporated herein by reference in its entirety.

Microbial fermentation is a biotechnological process through which bioactive peptides can be obtained. This process involves using microorganisms capable of producing proteolytic enzymes with the objective that these enzymes hydrolyze proteins into shorter peptides. The microorganisms generally used are bacteria, fungi, or yeasts, which may be present in the substrate indigenously or added as a starter culture. The microbial fermentation process can be divided into several systems. However, submerged fermentation and solid-state fermentation are the most widely used.

Submerged fermentation uses a culture of microorganisms in a liquid medium, which contains nutrients. This system is suitable for microorganisms with high water level activities, such as bacteria, and offers the advantage that the generated bioactive peptides are easy to purify. Solid-state fermentation uses microbial growth on nutrient-rich solid substrates. It has the advantage of releasing nutrients in a controlled way and is suitable for fungi and microorganisms with fewer moisture requirements.

Bioactive walnut peptides may be produced using any method known to those skilled in the art such as those disclosed in Merrifield, R. B., Solid Phase Peptide Synthesis I., J. AM. CHEM. SOC. 85:2149-2154 (1963); Carpino, L. A. et al., [(9-Fluorenylmethyl)Oxy] Carbonyl (Fmoc) Amino Acid Chlorides: Synthesis, Characterization, And Application To The Rapid Synthesis Of Short Peptides, J. ORG. CHEM. 37:51:3732-3734; Merrifield, R. B. et al., Instrument For Automated Synthesis Of Peptides, ANAL. CHEM. 38:1905-1914 (1966); or Kent, S. B. H. et al., High Yield Chemical Synthesis Of Biologically Active Peptides On An Automated Peptide Synthesizer Of Novel Design, IN: PEPTIDES 1984 (Ragnarsson U., ed.) Almqvist and Wiksell Int., Stockholm (Sweden), pp. 185-188, which are all incorporated herein by reference in their entirety.

The therapeutically effective amount of the one or more bioactive walnut peptides will vary depending on the bioactive walnut peptide and the combination of bioactive walnut peptides. Nonetheless, in various embodiments, the therapeutically effective amount may be from about 1 μg to about 50 mg (50,000 μg) per cm² of skin. In further embodiments, the therapeutically effective amount of the one or more walnut peptides is from about 1 μg to about 40 mg, about 1 μg to about 30 mg, about 1 to about 20 mg, about 1 μg to about 10 mg, about 1 μg to about 8,000 μg, about 1 μg to about 5,000 μg, about 1 μg to about 2,000 μg, about 1 μg to about 1,000 μg, about 10 μg to about 40 mg, about 10 μg to about 30 mg, about 10 to about 20 mg, about 10 μg to about 10 mg, about 10 μg to about 8,000 μg, about 10 μg to about 5,000 μg, about 10 μg to about 2,000 μg, about 10 μg to about 1,000 μg, about 100 μg to about 50 mg, about 100 μg to about 40 mg, about 100 μg to about 30 mg, about 100 μg to about 20 mg, about 100 μg to about 10 mg, about 100 μg to about 8,000 μg, about 100 μg to about 5,000 μg, about 100 μg to about 2,000 μg, about 100 μg to about 1,000 μg, about 500 μg to about 50 mg, about 500 μg to about 40 mg, about 500 μg to about 30 mg, about 500 μg to about 30 mg, about 500 μg to about 20 mg, about 500 μg to about 10 mg, about 500 to about 8,000 μg, about 500 μg to about 5,000 μg, about 500 μg to about 2,000 μg, or about 500 μg to about 1,000 μg per cm² of skin.

The bioactive walnut peptides are typically formulated with a physiologically acceptable carrier and applied to the skin as a pharmaceutical or cosmetic composition. The amount of the one or more bioactive walnut peptides will vary depending on their activity, use, and interaction with additional bioactive walnut peptides that may optionally be included in the pharmaceutical or cosmetic composition. Nonetheless, in various embodiments, the pharmaceutical or cosmetic composition include about 0.01 to about 10 wt. % of the one or more bioactive walnut peptides, based on the total weight of the pharmaceutical or cosmetic composition.

In further embodiments, the pharmaceutical or cosmetic composition include about 0.01 to about 8 wt. %, about 0.01 to about 5 wt. %, about 0.01 to about 3 wt. %, about 0.01 to about 1 wt. %, about 0.01 to about 0.5 wt. %, about 0.1 to about 10 wt. %, about 0.1 to about 8 wt. %, about 0.1 to about 5 wt. %, about 0.1 to about 3 wt. %, about 0.1 to about 1 wt. %, about 0.5 to about 10 wt. %, about 0.5 to about 8 wt. %, about 0.5 to about 5 wt. %, about 0.5 to about 3 wt. %, about 0.5 to about 2 wt. %, about 1 to about 10 wt. %, about 1 to about 8 wt. %, about 1 to about 5 wt. %, about 1 to about 3 wt. %, or about 0.1 to about 2 wt. % of the one or more bioactive walnut peptides, based on the total weight of the pharmaceutical or cosmetic composition.

In an embodiment, the one or more bioactive peptides and the pharmaceutical or cosmetic compositions described throughout the instant disclosure are useful for improving tensile properties of the skin, potentiating collagen synthesis, and/or activating the collagen biosynthesis pathway.

Collagen occurs in many places throughout the body. So far, at least 28 types of collagen have been identified and described which provide a variety of structural and functional properties that collagen exhibits throughout the body. The five most common types are collagen I, II, III, IV and V. However, over 90% of the collagen in the body is of type I.

In human skin, collagen types I and Ill are the predominant types of collagen. They are present as fibrils and are responsible for the solidity and the strength of the dermis. Since type I collagen is the predominant collagen in adult human skin, comprising about 80% of the total bulk of collagen, it plays a major role in providing tensile strength to the skin. It is clear, however, that type III collagen, which comprises some 10% of the total dermal collagen, also plays a critical role in providing additional tensile properties to the skin and other tissues.

Structurally, three collagen polypeptides wrap around each other in a helix to form a triple helix collagen I or III molecule. These molecules are packed in a five-stranded rope-like structure wherein each collagen molecule is quarter-staggered with respect to the next to form a microfibril. Microfibrils are subsequently wrapped around other microfibrils to form fibrils, which in turn wrap around other fibrils to produce even larger fibers.

Using histological and ultrastructural approaches in the past, it has been well-described that chronologically aged skin manifests in a reduced synthesis of both collagen types I and 11. With respect to photoaging, Schwarz et aL. (PHOTOCHEM PHOTOBIOL 1993, 58, 841-844) demonstrated that the loss in collagen in sun-damaged human skin is due to increased degradation of both types I and III collagens. Additionally, it was shown that fibroblasts derived from sun-exposed skin synthesize a lower proportion of collagen III than cells from sun-protected skin. (J PHOTOCHEM PHOTOBIOL B. 1995, 27: 33-38).

Other collagen types are also present in skin and can change with skin aging. For instance, collagen VII, which is responsible for anchoring the basement membrane onto dermal matrix, decreases with aging. (See EUR J DERMATOL 2008; 18: 297-302). One of the additional major morphological features of aged skin is an altered dermal epidermal junction which structurally manifests as flattening of the dermal epidermal junction outline with the loss of rete pegs and re-duplication of the lamina densa. Since the dermal epidermal junction is involved in the cohesion between the dermis and epidermis, age-related alterations in the dermal epidermal junction as a result of collagen VII decreasing results in functional changes in skin's resistance to mechanical stress and tissue homeostasis, which likely contributes to wrinkle formation.

Collagen V assembles into diverse molecular forms and is believed to be expressed in skin as different subtypes with important but distinct roles in matrix organization and stability. (See J INVEST DERMATOL 2012, 132: 1841-1849). Whereas collagen V is the defective product in most cases of classical Ehlers-Danlos syndrome, which is an extracellular matrix component disorder typically characterized by skin fragility and abnormal wound healing, it does not seem to significantly change with skin aging.

Further collagen types such as collagens VI, XIV and XVI are also expressed in the collagen-rich dermis.

The production of collagen in vivo requires activation of the collagen biosynthesis pathway by which transcription in the cell nucleus promotes polypeptide synthesis via translation from mRNA, organization of the polypeptides into a pro-collagen triple helix in the cytoplasm, secretion of pro-collagen from the cell, and then cleavage reactions, fibril assembly, and cross-linking extracellularly. Unlike many proteins that are stored in secretory granules and then secreted from the cell upon demand, collagen is secreted continuously.

In an embodiment of the instant disclosure, the one or more bioactive walnut peptides and the pharmaceutical or cosmetic compositions comprising them are useful for improving elasticity of the skin, potentiating elastin synthesis, and preventing elastin deterioration.

Elastin is a vital component of the extracellular matrix of vertebrates and provides exceptional properties including elasticity and tensile strength to many tissues and organs including the skin. Mature elastin is an insoluble and extremely durable protein that undergoes little turnover, but sustained exposure to proteases may lead to irreversible and severe damage, and thus to functional loss of the elastic fiber network. In general, elastin content decreased with age in sun unexposed skin (i.e., buttock) (i.e., elastin content decreased by about 44% between 50 and 70 years of age). A similar decrease was seen in severe sun-exposed skin (i.e., face) (i.e., elastin content decreased by about 31% between 50 and 70 years of age). Interestingly, the elastin content in moderately sun-exposed areas (i.e., forearm) did not significantly change during aging. This phenomenon might be explained by a combination of age-induced reduction and sun-dependent increase in elastin, what appears to be at least partially regulated by UV-induced deposition of lysozyme in elastin fibers. (See JEADV, 2006, 20, 980-987).

Fibrillins (e.g., fibrillin 1) are ubiquitous glycoproteins of the extracellular matrix that self-polymerize into filamentous microfibrils in which individual molecules are organized in longitudinal head-to-tail arrays and associate laterally as well. (See FIBROGENESIS & TISSUE REPAIR, 2010, 3, 24). Fibrillin microfibrils can additionally serve as the structural template for tropoelastin deposition and/or crosslinking during elastic fiber formation. Specific segments of the fibrillins interact in vitro with numerous extracellular signaling and cell surface molecules, including fibronectin, fibulins, syndecans and integrins. The multiple molecular interactions of fibrillins are believed to drive the assembly of morphologically distinct macroaggregates, which contribute to imparting the structural integrity to individual tissues and organs (structural role), and to target TGF-beta and BMP complexes to the architectural matrix, which contributes to instructing the behavior of cells (instructive role). TGF-betas and BMPs are potent modulators of extracellular matrix metabolism that are under the control of a complex network of relays and servomechanisms operating within and outside the cell, and at the cell surface. Thus, fibrillins are important components of the extracellular matrix which are necessary for the formation of other extracellular matrix components such as elastin and elastic fiber formation.

In an embodiment of the instant disclosure, the one or more bioactive peptides and the pharmaceutical or cosmetic compositions comprising them are useful for increasing hyaluronic acid levels in the extra cellular matrix of the skin, potentiating glycosaminoglycan production, or a combination thereof.

Hyaluronic acid (also called hyaluronan) is an anionic, non-sulfated glycosaminoglycan. It is unique among glycosaminoglycans since it is non-sulfated and can be very large, with its molecular weight (g mol-) often reaching the millions. As a predominant voluminous molecule, hyaluronic acid is a major component of the extracellular matrix of the skin. It provides structure, volume (which is associated with hyaluronic acid's excellent water holding properties), and organization (e.g., facilitates the transport of ion solutes and nutrients) but also contributes significantly to cell proliferation and migration in the dermis. In addition, through the water-attaining properties of hyaluronic acid, it contributes to the hydration of the skin.

Glycosaminoglycans (e.g., hyaluronic acid, chondroitin sulfate, heparan sulfate, dermatan sulfate, keratan sulfate, etc.) and particularly hyaluronic acid are major components of the cutaneous extracellular matrix involved in wound healing and tissue regeneration. Wound healing is a dynamic interactive process involving many precisely interrelated phases, overlapping in time, and leading to the restoration of tissue integrity. The healing process reflects the complex and coordinated body response to tissue injury resulting from the interaction of different cell types and extracellular matrix components. Hyaluronan plays a key role in each phase of wound healing by stimulating cell migration, differentiation, and proliferation as well as regulating extracellular matrix organization and metabolism. Glycosaminoglycans and particularly hyaluronic acid are also involved in skin aging.

As summarized by R. Stern in 2010 (TEXTBOOK OF AGING SKIN, Springer), though dermal hyaluronic acid is responsible for most hyaluronic acid in skin, epidermal cells (e.g. keratinocytes) are also able to synthesize hyaluronic acid. The most dramatic histochemical change observed in aged skin is the marked decrease in epidermal hyaluronic acid. In skin of an elderly person, hyaluronic acid is still present in the dermis, while the hyaluronic acid of the epidermis has disappeared entirely. The reasons for this precipitous fall with aging are unknown. It has been described that the synthesis of epidermal hyaluronic acid is influenced by the underlying dermis, thereby indicating that epidermal hyaluronic acid is under separate controls from dermal hyaluronic acid.

In an embodiment of the instant disclosure, the one or more bioactive peptides and the pharmaceutical or cosmetic compositions comprising them are useful for wound healing, minimizing scarring, or a combination thereof.

Other than in skin aging, collagens I and III are also major extracellular matrix components involved in scar formation. Scarring occurs after trauma, injury or surgery to any tissue or organ in the body. Such scars are a consequence of a repair mechanism that replaces the missing normal tissue with an extracellular matrix consisting predominantly of collagen types I and Ill as well as fibronectin and some other extracellular matrix components. Scarring represents imperfect tissue regeneration. Whereas skin wounds on early mammalian embryos (e.g., up to about 24 weeks of gestation in humans) heal perfectly with no signs of scarring and complete restitution of the normal skin architecture, postnatal wounds heal with scars. (See Dang C et aL., CLIN PLAST SURG 2003: 30, 13-23).

There are phenotypic differences between the collagen content and cross-linking patterns in fetal and postnatal wounds (See CLIN PLAST SURG 2003, 30, 13-23 and CURR OPIN PEDIATR 2012, 24, 371-378). In fetal wounds, type I∥ collagen is rapidly deposited in a fine reticular network that is indistinguishable from uninjured skin. Post-natal, the ratio of type I to type 11 collagen in wounds increases. Of the many different types of collagen identified, fetal skin is known to contain a greater proportion of type I1 collagen, whereas adult skin consists predominantly of type I collagen. The predominance of type I collagen in postnatal wounds provides regenerating tissue with more strength and rigidity. Early scar formation in late gestation fetal wounds demonstrates larger collagen fibers with greater inter-fiber space.

In an embodiment of the instant disclosure, the one or more bioactive peptides and the pharmaceutical or cosmetic compositions comprising them are useful for preventing, treating, or reversing skin aging and/or signs of skin aging.

All terms such as “skin aging,” “signs of skin aging,” “topical application,” and the like are used in the sense in which they are generally and widely used in the art of developing, testing, and marketing pharmaceutical, cosmetic, and personal care products, as well as for medicaments which are indicated for skin aging.

Skin aging is classified into intrinsic aging and extrinsic aging depending on its cause. Intrinsic aging is a process by which the skin structure and the physiological functions of the skin deteriorate regardless of environmental changes as a human gets older. Extrinsic aging is caused by continuous exposure to external environment such as sunlight and air pollutants. Especially, skin aging caused by sun light is called photoaging. Ultraviolet (UV) light from the sun is the main cause of the physiological and morphological changes in aged skin.

As intrinsic skin aging proceeds, the skin becomes dry, and fine lines and wrinkles form which become more visible and deepen with age. Further, because of structural and functional changes of the epidermis and the dermis, the skin loses its elasticity and looks drooping. The dermis becomes thinner and well visible skin folds (e.g., nasolabial fold) form with age. It is estimated that the total quantity of collagen lost each year for adults is about 1%. In addition, the remaining collagen fibers gradually become thicker, while the cross-linking of the collagen fibers increases, so that the solubility, elasticity and like thereof decrease. Furthermore, elastin fibers become thicker, and the cross-linking thereof also increases. Moreover, the proliferative activity of fibroblasts in the dermis decreases with time, and the ability of the aging fibroblasts to form (i.e., synthesize) new collagen, elastin, hyaluronic acid, and other components of the extracellular matrix also decreases.

Continuous exposure to the sun is the main cause of extrinsic aging of skin. The UV component of sunlight, particularly UVA and UVB, is generally believed to be the principal causative agent in this process called photoaging. The extent of UV exposure required to cause “photoaging” is not currently known, although the amount sufficient to cause erythema (reddening, commonly described as sunburn) in human skin is quantified as the “minimal erythema dose” (MED) from a given UV light source. Repeated exposure to sunlight UV at levels that cause erythema and tanning are, nevertheless, commonly associated with photoaging.

There is a difference between the physiology of intrinsically aged (i.e., chronologically aged) skin in comparison with that of photoaged skin. Chronologically aged skin typically maintains a smooth and unblemished appearance, in comparison with the leathery, blotchy, and often deep wrinkling of photoaged skin. Photoaging is characterized clinically by coarseness, wrinkles, mottled pigmentation, sallowness, laxity, telangiectasia, lentigines, purpura and relative ease of bruising, atrophy, depigmented areas, eventually premalignant, and ultimately malignant neoplasm (i.e., abnormal mass of tissue due to neoplasia, which is the abnormal proliferation of cells). Photoaging commonly occurs in skin that is generally exposed to sunlight such as the face, ears, bald areas of the scalp, neck, decollets, forearms, and hands.

Although the typical appearance of photoaged and chronologically aged human skin can be readily distinguished, recent evidence indicates that chronologically aged and UV-irradiated skin share important molecular features including altered signal transduction pathways that promote matrix-metalloproteinase expression (e.g., collagenase, gelatinase) causing extracellular matrix degradation, decreased collagen formation, and alteration or damage to extracellular matrix of skin such as the accumulation of amorphous elastin-containing material residing beneath the epidermal dermal junction. This concordance of molecular mechanisms suggests that UV irradiation accelerates many key aspects of the chronological aging process in human skin.

“Signs of skin aging” include, but are not limited to, all outward visibly and tactilely perceptible manifestations as well as any other macro or micro effects due to skin aging. Such signs may be induced or caused by intrinsic factors (showing as chronological aged skin) and extrinsic factors (showing as environmental skin damage including but not limited photo-aged skin). These signs may result from processes which include, but are not limited to, the development of textural discontinuities such as wrinkles and coarse deep wrinkles, fine or skin lines, crevices, bumps, large pores (e.g., associated with adnexal structures such as sweat gland ducts, sebaceous glands, etc.), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including under eye circles), blotching, sallowness, hyperpigmented skin regions such as age spots and freckles, keratoses, abnormal differentiation, hyper-keratinization, elastosis, collagen breakdown, and other histological changes in the stratum corneum, dermis, epidermis, the skin vascular system (e.g., telangiectasia or spider vessels), and underlying tissues, especially those proximate to the skin.

In various embodiments of the instant disclosure, the one or more bioactive peptides and the pharmaceutical or cosmetic compositions comprising them are useful for prophylactically preventing, treating, or regulating a skin condition. As used herein, prophylactically preventing, treating, or regulating a skin condition includes delaying, minimizing and/or preventing visible and/or tactile discontinuities in skin (e.g., texture irregularities in the skin which may be detected visually or by feel), including signs of skin aging. It includes ameliorating, e.g., diminishing, minimizing and/or effacing, discontinuities in skin, including signs of skin aging.

In further embodiments, the bioactive walnut peptides are useful in one or more methods for depigmenting the skin, lightening the skin, brightening the skin, treating hyperpigmentation, and treating melasmic skin. Similarly, the bioactive walnut peptides are useful in methods for evening out skin tone. The bioactive walnut peptides or compositions comprising them can be applied to skin identified as having hyperpigmentation, melasmic skin, sunspots, aged spots, discolored spots, skin having uneven skin tone, etc. In particular embodiments there is disclosed is a method of lightening skin or evening skin tone comprising applying one or more bioactive walnut peptides or a cosmetic composition comprising the one or more bioactive walnut peptides to the skin. The method can further comprise identify a person in need of skin lightening or evening skin tone. The methods can further include inhibiting melanogenesis in a skin cell, inhibiting tyrosinase or tyrosinase synthesis in a skin cell, or inhibiting melanin transport to keratinocytes in a skin cell. The one or more bioactive walnut peptides or a cosmetic composition comprising the one or more bioactive walnut peptides can act as an alpha melanin stimulatory hormone antagonist. The one or more bioactive walnut peptides or a cosmetic composition comprising the one or more bioactive walnut peptides can even out pigmentation of the skin. In nonlimiting aspect, lightening skin can include reducing the appearance of an age spot, a skin discoloration, or a freckle by topical application of the composition to skin having an age spot, skin discoloration, a freckle, etc.

Also disclosed is a method of treating hyperpigmentation comprising applying one or more bioactive walnut peptides or a cosmetic composition comprising the one or more bioactive walnut peptides to the skin. The method can also comprise identifying a person in need of treating hyperpigmentation. Additional methods contemplated include methods for reducing the appearance of an age spot, a skin discoloration, or a freckle, reducing or preventing the appearance of fine lines or wrinkles in skin, or increasing the firmness of skin.

In another embodiment there is disclosed methods for reducing the appearance of uneven skin tone comprising topically applying one or more bioactive walnut peptides or a cosmetic composition comprising the one or more bioactive walnut peptides to skin. The uneven skin tone can be caused by discolored skin. The one or more bioactive walnut peptides or a cosmetic composition comprising the one or more bioactive walnut peptides can be applied to discolored skin (e.g., facial skin, arm skin, leg skin, scalp, neck skin, chess skin, abdomen skin, hand skin, etc.). The discolored skin can be an age spot, blotchy skin, a freckle, hyperpigmented skin, skin suffering from melasma, skin that has been over-exposed to sun, etc. The method can also be used to improve a person's skin tone by topical application of the one or more bioactive walnut peptides or a cosmetic composition comprising the one or more bioactive walnut peptides disclosed throughout this specification to skin that has discolored skin. The method can also be used to prevent the appearance of uneven skin tone by topical application of the compositions disclosed throughout this specification to skin that is at risk of developing uneven skin tone. Skin at risk of developing uneven skin tone includes skin that has been over-exposed to sun, pregnant women, people having or at risk of developing melasma, post-inflammatory hyperpigmentation (e.g., darkening of skin after injury to skin such as an acne lesion or a burn). The one or more bioactive walnut peptides or a cosmetic composition comprising the one or more bioactive walnut peptides of the present invention can also be used to lighten skin.

Pharmaceutical and cosmetic compositions including the one or more bioactive walnut peptides can be formulated in various forms, for example, lotions, creams, serums, sprays, emulsions, gels, powders, dispersions, ointments, sticks, pastes, and foams. In addition to a physiologically acceptable carrier, the pharmaceutical and cosmetic composition may optionally include one or more of the following:
Ascorbates and other vitamins,
Sunscreen active agents,
Anti-wrinkle active agents and anti-atrophy active agents,
Wetting agents, humectants, and conditioning agents,
Emollients,
Antioxidants and free radical scavengers,
Desquamating active agents, keratolytic agents and exfoliating agents,
Suspending agents,
Emulsifying agents,
Thickeners, and/or
Antihistamines.

### Ascorbates and Other Vitamins

The pharmaceutical or cosmetic compositions of the instant disclosure may contain one or more vitamins, such as ascorbates (for example, vitamin C, vitamin C derivatives, ascorbic acid, ascorbyl glucoside, ascorbyl palmitate, magnesium ascorbyl phosphate, sodium ascorbyl phosphate, tetrahexadecyl scorbate, ascorbyl phosphate, aminopin-3-aminopinamine) B, derivatives of vitamin B, vitamin B1-vitamin B12 and their derivatives, vitamin K, derivatives of vitamin K, vitamin H, vitamin D, vitamin D3, derivatives of vitamin D, vitamin E, derivatives of vitamin E and its provitamins, such as ntenol, and mixtures thereof. Vitamin compounds may be included as a substantially pure material or as an extract obtained by suitable physical and/or chemical isolation from natural (eg, plant) sources.

The amount of the one or more vitamin compounds in the pharmaceutical or cosmetic compositions will vary but are typically from about 0.0001% to about 25%, more preferably from about 0.001% to about 10%, more preferably from about 0.01% to about 5%, and more preferably from about 0.1% to about 1%, based on the total weight of the pharmaceutical or cosmetic compositions.

### Sunscreen Active Agents

The pharmaceutical or cosmetic composition of the instant disclosure may optionally contain sunscreen active agents, which can also be referred to as a UV filtering agent. As used herein, “sunscreen active agents” include both sun reflectors and physical sunscreen agents. Suitable sunscreen active agents may be organic or inorganic. A wide variety of standard organic or inorganic sunscreen active agents are suitable for use in accordance with the present invention.

The amount of the one or more sunscreen active agents in the pharmaceutical or cosmetic compositions will vary. Nonetheless, in certain embodiment, the pharmaceutical or cosmetic compositions include from about 0.1% to about 25%, more often from about 0.5% to about 10%, of sunscreen active agents, based on the total weight of the pharmaceutical or cosmetic compositions.

Nonlimiting examples of organic sunscreen active agents include ethylhexyl salicylate, butyl methoxybenzoylmethane, ethylhexylmethoxycinnamate, octocrylene, phenylbenzimidazole sulfonic acid, terephthalilide dicamphora sulfonic acid, benzophenone-4-benzophenone-3 camphor methylbenzylidene, benzimidazylate, anisotriazine, ethylhexyltriazone, diethylhexylbutamidotriazone, methylene-bis-benzotriazolyltetramethylbutylphenol, dromethrazole trisiloxane, and mixtures thereof.

Nonlimiting examples of inorganic sunscreen agents include nanopigments (with an average primary particle size: generally, between 5 nm and 100 nm, preferably between 10 nm and 50 nm), or aggregates, whether or not coated with metal oxides, such as, for example, titanium oxide nanopigments (amorphous or crystalline in the form of rutile and/or anatase), iron, zinc, zirconium or cerium oxides, and mixtures thereof. Coating agents are also alumina and/or aluminum stearate and silicones.

### Anti-Wrinkle Active Agents and Anti-Atrophy Active Agents

The pharmaceutical or cosmetic compositions of the instant disclosure may optionally include one or more anti-wrinkle active agents or anti-atrophy active agents.

Nonlimiting examples of anti-wrinkle active agents and anti-atrophy active agents include proxylane, amino acids, N-acetyl derivatives of amino acids (e.g., N-acetylcysteine), hydroxyl acids (e.g., alpha hydroxy acids, such as lactic acid and glycolic acid, or beta-hydroxy acids, such as salicylic acid and salicylic acid derivatives, such as octanoyl derivative, lactobionic acid), keto acids (e.g. pyruvic acid), fiti hydrochloric acid, ascorbic acid (vitamin C), retinoids (e.g. retinoic acid, tretinoin, isotretinoin, adapalene, retinol, retinylaldehyde, retinyl palmitate and other retinoid derivatives), kinetin (N6-furfuriladenenin), zeatin, and their derivatives, for example, furunyrenifenyrenifenyrnidirophenide, niacinamide (nicotinamide); growth factors and cytokines (for example, TGF-beta 1, 2 and 3, ERF, FRF-2, TGF, IL-1, IL-6, IL-8, IGF-1, IGF-2, etc.), cell lysates (e.g., cell lysate of dermal fibroblasts, stem cell lysate, treated skin cell proteins (PSP®), etc.), conditioned culture media (e.g., conditioned culture medium from dermal fibroblasts, conditioned culture medium from stem cells (e.g., stem cells of the epidermis, stem cells of adipose tissue, mesenchymal stem cells, etc.); cosmetic products sold under the trade names Nouricel-MD®, TNS® or CCM™ Complex; etc.); cell extracts, stem cell extracts, stem cell components; ingredients stimulating epidermal or other adult stem cells; skin conditioning agents, stilbenes, cinnamates, sirtuin 1 activating ingredients (e.g., resveratrol); mitochondrial function enhancing ingredients; dimethylaminoethanol, synthetic anti-aging peptides, peptides from natural sources (e.g., soy peptides) and sugar salts (e.g., Mn gluconate, Zn gluconate), lipoic acid; lysophosphatidyl acid, vitamin B3 compounds and other vitamin B compounds (e.g. thiamine (vitamin B1), pantothenic acid (vitamin B5), riboflavin (vitamin B2) and their derivatives and salts (e.g., HCl salts or calcium

The total amount of anti-wrinkle and/or anti-atrophy active agents in the pharmaceutical or cosmetic compositions will vary. Nonetheless, in certain embodiments, the pharmaceutical or cosmetic compositions include from about 0.0001% to about 10%, more preferably from about 0.001% to about 8%, more preferably from about 0.01% up to about 5% and more preferably from about 0.1% to about 1% of the one nor more anti-wrinkle and/or anti-atrophy active agents by weight, based on the total weight of the composition.

### Wetting Agents, Humectants, and Conditioning Agents

The pharmaceutical or cosmetic composition of the present invention may optionally include an effective amount a wetting agent, a moisturizer, and/or a skin conditioning agent. The total amount of the one or more wetting agents, humectants, and conditioning agents, if present, will vary. Nonetheless, in certain embodiments, the pharmaceutical or cosmetic compositions include from about 0.01% to about 80%, more preferably from about 0.1% to about 25% and more preferably from about 0.5% to about 10% weight of the one or more wetting agents, humectants, and/or conditioning agents, based on the total weight of the pharmaceutical or cosmetic composition.

Wetting agents are ingredients that help maintain moisture levels in the skin. Nonlimiting examples of wetting agents include polyhydric alcohols, water soluble alkoxylated nonionic polymers and mixtures thereof. Nonlimiting examples of polyols include the polyhydroxy alcohols listed above and glycerol, hexylene glycol, ethoxylated glucose, 1,2-hexanediol, dipropylene glycol, trehalose, diglycerin, maltitol, maltose, glucose, fructose, sodium chondrophosphate gum sodium nitrophosphate, sodium lactate, pyrrolidone carbonate, glucosamine, cyclodextrin and mixtures thereof. Water-soluble alkoxylated non-ionic polymers useful in this application include polyethylene glycols and polypropylene glycols, the molecular weight of which is up to about 1000, such as polyethylene glycols and polypropylene glycols, named in accordance with the Association for Perfumes and Cosmetics and Fragrances (CTFA) PEG-200, PEG-400, PEG-600, PEG-1000, and mixtures thereof. Additional wetting agents include acetylarginine, algae extract, aloe barbaden leaf extract, 2,3-butanediol, chitosan lauroylglycinate, diglyceret-7 malate, diglycerol, diglycol guanidine succinate, erythritol, fructose, glucose glycosyl hydrochloride, glycosyl hydrochloride, glycosyl hydrochloride, glycosyl hydrochloride, glycosyl hydrochloride, inositol, lactitol, maltitol, maltose, mannitol, mannose, methoxypolyethylene glycol, myristamidobutyl guanidine acetate, polyglyceryl sorbitol, potassium pyrrolidone carboxylic acid (PCA), propylene glycol, butylene glycol, atria pyrrolidone carboxylic acid (PCA), sorbitol, sucrose, dextran.

Nonlimiting examples of conditioning agents guanidine, urea, glycolic acid, glycolates (eg, ammonium and quaternary alkylammonium), salicylic acid, lactic acid, lactates (e.g., ammonium and quaternary alkylammonium), aloe vera any of its various forms (e.g. aloe vera gel), polyhydroxy alcohols such as sorbitol, mannitol, xylitol, erythritol, hexanetriol, butanetriol, propylene glycol, butylene glycol, hexylene glycol, etc., polyethylene glycols, propoxylated glyce alternatives, sugars (e.g., melibioses), starches, sugar and starch derivatives (e.g., alkoxylated glucose, fructose, glucosamine), C1-C30 monoesters and polyesters of sugars and related materials, hyaluronic acid, lactamide monoethanolamine, acetamide monoanthenol, acetamide monoanthenol allantoin and mixtures thereof. Skin conditioning agents may also include fatty acids, fatty acid esters, lipids, ceramides, cholesterol, cholesterol esters, beeswax, petroleum jelly, and mineral oil.

### Emollients

One or more emollients may also optionally be included in the pharmaceutical or cosmetic composition described herein. An emollient generally refers to an ingredient that can help maintain a soft, smooth, and supple skin appearance. Emollients generally remain on the skin surface or in the stratum corneum and act as a moisturizer or lubricant and reduce delamination. Nonlimiting examples of emollients include acetylarginine, acetylated lanolin, algal extract, polyethylene glycol-6 esters from apricot kernel oil, polyethylene glycol-11 esters from avocado oil, bis-polyethylene glycol-4 dimethicone, butoxyethyl stearate, glycol esters, alkyl glycol ethers esters, cetyl laurate, coconut polyethylene glycol-10 esters, alkyl tartrates, diethyl sebacate, dihydrocholesteryl butyrate, dimethiconol, dimyristyl tartrate, distearare-5 lauroylg utamat, etilavokadat, ethylhexyl myristate, glyceryl isostearate, glyceryl oleate, geksildetsilstearat, geksilizostearat, hydrogenated palm glycerides, hydrogenated soy glycerides, hydrogenated glycerides of fat izostearilneopentanoat, isostearyl palmitate, izotridetsilizononanoat, laureth-2 acetate, lauryl polyglyceryl-6 cetearyl glycol ether, metilglyutset-20 benzoate, mineral oil, palm oil, coconut oil, miret-3 palmitate, octyldecanol, octyldodecanol, Odontella aurita oil, 2-oleamido-1,3 octadecandiol, pal commercial glycerides, glycerides of polyethylene glycols avocado, polyethylene glycol castor oil, copolymer of polyethylene glycol-2/dodecyl glycol, glycerides of polyethylene glycol shea butter, phytol, raffinose, stearyl citrate, glycerides of sunflower seed oil, non-ointment, small tocopheryl glucoside.

### Antioxidants and Free Radical Scavengers

The pharmaceutical or cosmetic composition of the present invention may optionally include one or more antioxidants and/or free radical scavengers. For example, the compositions may include from about 0.001% to about 10%, more preferably from about 0.01% to about 8%, and more preferably from about 0.1% to about 5% of the one or more antioxidants and/or free radical scavengers, based on the total weight of the composition.

Nonlimiting examples of antioxidants and scavengers of free radicals include falcata bark extract, ascorbic acid (Vitamin C) and its salts, fatty acid ascorbyl esters and other derivatives of ascorbic acid (for example, magnesium ascorbyl phosphate, sodium ascorbyl phosphate, ascorbyl sorbate, ascorbyl tetrabeplite, tetra ascorbyl tetrabal and ascorbyl tetraxalde etc.), tocopherol (vitamin E), tocopherolsorbate, tocopherol acetate, other tocopherol esters, beta-carotene, butylated hydroxybenzoic acids and their salts, ferulic acid, peroxides, including hydrogen peroxide, perborate, thioglycolates, persulfate salts, 6-hydroxy-2,5,7,8-tetramethylchroman-2-carboxylic acid (commercially available under the trade name Trolox™) gallic acid and its alkyl esters, in particular propyl gallate, uric acid and its salts and alkyl esters, amines (e.g. N, N-diethylhydroxylamine, aminoguanidine), nordihydroguayaretic acid, bioflavonoids, sulfhydryl compounds (e.g. glutathione), dihydroxyfumaric acid and its salts, lysine ligninphenolate silymar in, lysine, 1-methionine, proline, superoxide dismutase, sorbic acids and their salts, lipoic acid, olive extracts, tea extracts, resferatrol, polyphenols such as Bunge proanthrocyanidin, carotenoids, curcumin compounds such as tetrahydrocurcumin, coenzyme Q10 (L-2-oxo-4-thiazolidinecarboxylic acid), selenium, creatine, glutathione, N-acetylcysteine, N-acetylcysteine esters, dimethylmethoxychromanol, lipoic acid, melanin; plant extracts containing polyphenols, including, but not limited to, coffee bean extracts, green tea extracts, rosemary extracts, witch hazel extracts, and grape skin/seed extracts that can be used. Preferred free radical antioxidants/scavengers may be selected from esters of ascorbic acid, tocopherol, ferulic acid, polyphenols, creatine and their derivatives; as well as plant extracts containing polyphenols, such as green tea extract.

### Suspending Agents

The pharmaceutical or cosmetic composition of the present invention may optionally include one or more suspending agents, preferably in a concentration effective to suspend the water-insoluble material in a dispersed form in the compositions or to modify the viscosity of the composition. Such concentrations will vary. Nonetheless, in certain embodiments, the pharmaceutical and cosmetic composition includes from about 0.1% to about 10%, more preferably from about 0.25% to about 5.0% of the one or more suspending agents, based on the total weight of the composition. Nonlimiting examples include vinyl polymers, such as cross-linked acrylic acid polymers, called carbomer, cellulose derivatives and modified cellulose polymers such as methyl cellulose, ethyl cellulose, nitrocellulose, carboxymethyl cellulose, crystalline cellulose, cellulose powder, polyvinylpyrrolidone, polyvinyl alcohol, guar gum, hydroxypropyl guar gum, gum Arabic, galactan, locust bean gum, pectin, agar, starch (rice, corn, potato, wheat), algal colloids (algae extract), microbiological polymers such as dextran, succinoglycan, pullulan, starch-based polymers such as carboxymethyl starch, methyl starch, alginic acid polymers such as sodium alginate, alginic acid propylene glycol esters, acrylate polymers such as sodium polyacrylate, polyacrylate, polyacrylamide, polyethyleneimine and inorganic minutes water soluble material such as bentonite, aluminum magnesium silicate, laponite, hectonite, and anhydrous silicic acid.

Other optional suspending agents include crystalline suspending agents that can be resolved into acyl derivatives, long chain amine oxides, long chain acyl derivatives, and mixtures thereof. Said preferred suspending agents include fatty acid ethylene glycol esters, fatty acid alkanolamides, long chain fatty acid esters (for example, stearyl stearate, cetyl palmitate, etc.); long chain esters of long chain alkanolamides (for example, stearamide diethanolamide distearate, stearamide monoethanol amide stearate); and glyceryl esters (e.g., glyceryl distearate, trihydroxystearin, tribhengen). Other suitable suspending agents include primary amines containing a fatty alkyl fragment containing at least about 16 carbon atoms, examples of which include palmitamine or stearamine, and secondary amines containing two fatty alkyl fragments, each of which contains at least about 12 carbon atoms examples of which include dipalmitoylamine or di (hydrogenated fat) amine. Other suitable suspending agents include phthalic acid di amide (hydrogenated fat) and a crosslinked maleic anhydride/methyl vinyl ether copolymer.

### Emulsifying Agents

Nonlimiting examples of emulsifying agents include condensation products of alkylene oxides with fatty acids (ie alkylene oxide fatty acid esters), condensation products of alkylene oxides with 2 moles of fatty acids (ie fatty acid alkylene oxide diesters), condensation products alkylene oxides with fatty alcohols (ie fatty alcohol alkylene oxide esters), condensation products of alkylene oxides with both fatty acids and fatty alcohols [i.e. where a portion of the polyalkylene oxide is esterified at one end with a fatty acid and esterified (ie, via an ether bond) at the other end with a fatty alcohol]. Non-limiting examples of non-ionic surfactants derived from said alkylene oxide include cetet-6, cetet-10, cetet-12, cetetaret-6, cetetaret-10, cetetaret-12, stearet-6, stearet-10, stearet-12, stearet-21, PEG-6 stearate, PEG-10 stearate, PEG-100 stearate, PEG-12 stearate, PEG-20 glyceryl stearate, PEG-80 glyceryl tallowat, PEG-10 glyceryl stearate, PEG-30 glyceryl cocoate, PEG-80 glyceryl coco, PEG-200 glyceryl tallowat, PEG-8 dilaurate, PEG-10 distearate and mixtures thereof. Other applicable non-ionic surfactants include polyhydroxyamide fatty acid surfactants. A particularly preferred surfactant corresponding to the above structure is N-methylglucoside coconut alkylamide. Preferred among nonionic surfactants are surfactants selected from the group consisting of stearet-21, ceteareth-20, ceteareth-12, sucrose cocoate, stearet-100, PEG-100 stearate and mixtures thereof. Other non-ionic surfactants suitable for use in this application include sugar esters and polyesters, alkoxylated sugar esters and polyesters, C1-C30 fatty acid esters C1-C30 fatty alcohols, alkoxylated C1-C30 ester derivatives C1-C30 fatty alcohol fatty acids, alkoxylated C1-C30 fatty alcohol esters, polyglyceryl C1-C30 fatty acid esters, C1-C30 polyol esters, C1-C30 polyol esters, alkyl phosphates, polyoxyalkylene fatty ether forfates, fatty acid amides, acylactylates and mixtures thereof. Non-limiting examples of these emulsifiers include: polyethylene glycol 20 sorbitan monolaurate (polysorbate 20), polyethylene glycol 5 soy sterol, stearet-20, cetearet-20, PPG-2 methyl glucose ether distearate, cetet-10, polysorbate 80, cetyl phosphate, cetyl phosphate, cetyl phosphate, cetyl phosphate, cetyl phosphate, polysorbate 60, glyceryl stearate, polyoxyethylene 20 sorbitan triolcat (polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 and their PEG-100. Another group of non-ionic surfactants useful herein is a mixture of fatty acid esters based on a mixture of sorbitan or a sorbitol fatty acid ester and a sucrose fatty acid ester, where the fatty acid in each example is preferably C8-C24, more preferably C10-C20 fatty acid.

### Thickeners

Thickeners suitable for inclusion in the pharmaceutical or cosmetic composition described herein. Nonlimiting examples include acrylamide copolymer, agarose, amylopectin, bentonite, calcium alginate, calcium carboxymethyl cellulose, carbomer, carboxymethylchitin, cellulose gum, dextrin, gelatin, hydrogenated hydroxymethyl hydroxy cellulose hydroxypropyl, hydroxyethyl hydroxypropyl hydroxypropyl, hydroxyethyl hydroxypropyl, hydroxyethyl hydroxypropyl, hydroxyethyl hydroxypropyl, hydroxyethyl hydroxypropyl, hydroxyethyl hydroxypropyl, hydroxypropyl, hydroxypropyl, hydroxypropyl, hydroxypropyl, hydroxypropyl; magnesium alginate, methyl cellulose, microcrystalline cellulose, pectin, various polyethylene glycols, polyacrylic acid, polymethacrylic acid, polyvinyl alcohol, various sexes ipropylene glycols, copolymers of sodium acrylates, sodium carrageenan, xanthan gum and/or yeast beta-glucan, or mixtures thereof.

More generally, carboxylic acid polymers are useful thickeners. Polymers of carboxylic acids are crosslinked compounds containing one or more monomers derived from acrylic acid, substituted acrylic acids and salts and esters of said acrylic acids and substituted acrylic acids, wherein the crosslinking agent contains two or more carbon-carbon double bonds and comes from polyhydric alcohol. Examples of commercially available carboxylic acid polymers useful herein include carbomers, which are homopolymers of acrylic acid crosslinked with sucrose or pentaerythrotol allyl ethers. Carbomers are available as Carbopol® 900 Series from B.F. Goodrich (e.g. Carbopol® 954). In addition, other suitable carboxylic acid based polymeric agents include copolymers of C10-30 alkyl acrylates with one or more monomers of acrylic acid, methacrylic acid or esters of one of its short chains (i.e., C1-4 alcohol), the crosslinking agent is a sucrose or pentaerythritol allyl ether. These copolymers are known as acrylate/C10-30 alkyl acrylate crosspolymers and are commercially available as B.F. Carbopol® 1342, Carbopol® 1382, Pemulen TR-1 and Pemulen TR-2. Goodrich. Examples of preferred thickeners based on carboxylic acid polymers useful in this application include thickeners selected from carbomers, acrylate/C10-30 alkyl acrylate cross-polymers and mixtures thereof.

Moreover, according to certain embodiments, the thickeners are selected from polysaccharides. Nonlimiting examples of polysaccharide thickeners include cellulose, carboxymethyl hydroxyethyl cellulose, cellulose acetate propionate carboxylate, hydroxyethyl cellulose, hydroxyethyl cellulose, hydroxypropyl cellulose, hydroxypropyl methyl cellulose, sodium methyl hydroxyethyl cellulose hydroxyethyl cellulose and hydroxyethyl methyl hydroxyethyl hydroxyethyl cellulose hydroxyethyl methyl hydroxyethyl hydroxyethyl cellulose hydroxyethyl hydroxyethyl cellulose hydroxyethyl methyl hydroxyethyl hydroxyethyl. Alkyl-substituted celluloses are also useful. In these polymers, the hydroxy groups of the cellulosic polymer are hydroxylated (preferably hydroxyethylated or hydroxypropylated) to form hydroxylated cellulose, which is then further modified with a C10-30 straight or branched chain alkyl group via an ether bond. Typically, these polymers are straight chain or branched chain esters of C10-30 alcohols and hydroxyalkyl celluloses. Examples of alkyl groups applicable in this application include groups selected from stearyl, isostearyl, lauryl, myristyl, cetyl, isocetyl, cocoyl (e.g., an alkyl group derived from coconut oil alcohols), palmityl, oleyl, linoleil, linolenyl, recinoleil, behenyl and mixtures thereof. A preferred alkyl hydroxyalkyl cellulose ester is a material called cetyl hydroxyethyl cellulose, which is an ester of cetyl alcohol and hydroxyethyl cellulose, in accordance with the Perfume and Cosmetics and Perfume Association (CTFA). The indicated material is sold under the trade name Natrosol® CS Plus from Aqualon Corporation (Wilmington, Delaware). Further examples can be found in The International Cosmetic Ingredient Dictionary and Handbook, the Cosmetic Bench Reference—Directory of Cosmetic Ingredients, offered by the United States Pharmacopeia (USP) and National Formulary (NF), and other references to cosmetic and pharmaceutical ingredients known in the art. technicians. Other useful polysaccharides include scleroglucans, which are a straight chain (1-3) linked glucose units (1-6), where every three glucose units are linked, a commercially available example of which is Clearogel™ CS11 from Michel Mercier Products Inc. (Mountainside, New Jersey).

Other useful thickeners include those derived from natural sources. Nonlimiting examples include gum arabic, agar, algin, alginic acid, ammonium alginate, amylopectin, calcium alginate, calcium carrageenan, carnitine, carrageenan, dextrin, gelatin, gellan gum, guar gum hydrochloride, gidrohydrochloride, gidrohydrochloride, gidrohydrochloride, hydrochloride hyaluronic acid, hydrated silicon dioxide, hydroxypropylchitosan, hydroxypropyl gum, karaya gum, kelp, fruit tree resin, natto gum, potassium alginate, potassium carr gene, propylene glycol alginate, sclerotium gum, sodium carboxymethyl dextran, dextran, sodium carrageenan, tragacanth gum, xanthan gum and/or mixtures thereof.

In addition, the compositions may optionally contain polyacrylamide polymers, in particular nonionic polyacrylamide polymers, including substituted branched or unbranched polymers. Other polyacrylamide polymers useful herein include multi-block copolymers of acrylamides and substituted acrylamides with acrylic acids and substituted acrylic acids.

### Antihistamines

Antihistamines, also called histamine antagonists, are substances that inhibit the action of histamine by blocking its attachment to histamine receptors or by inhibiting the enzymatic activity of histidine decarboxylase, which catalyzes the conversion of histidine to histamine and the like. Nonlimiting examples of antihistamines are acrivastine, azelastine, brompheniramine, buclizine, bromodiphenhydramine, carbinoxamine, cetirizine, chlorpromazine, cyclizine, chlorpheniramine, chlorodiphenhydramine, cimetidine, clemastine, cyproheptadine, desloratine dimendenine dimendenine dimendenine dimendenine dimendenine dimendenine dimendenine dimendenine dimendenine dimenhydramin dimendenine dimendenine dimendenine dimendenine dimendenine dimendenine dimendenine dimendenine dimendenine dimendenine dimendenine dimendenine dimen dimerdenine dimen dimendenine dimenhydramine ebastine, embramine, famotidine, fexofenadine, lafutidine, levocetirizine, loratadine, meclosine, mirtazapine, nizatidine, olopadadine, orphenadrine, phenindamine, pheniramine, phenyltoloxamine, prometh zine, pyrilamine, quetiapine, ranitidine, roxatidine, rupatadin, tripelennamine, and triprolidine.

### EXAMPLES

Various changes can be made in the above-described compositions and methods without departing from the scope of the invention. Accordingly, it is intended that all disclosure contained in the above description and in the examples given below, shall be interpreted as illustrative and not in a limiting sense.

### Example 1

### Reduction of Inflammatory Cytokines

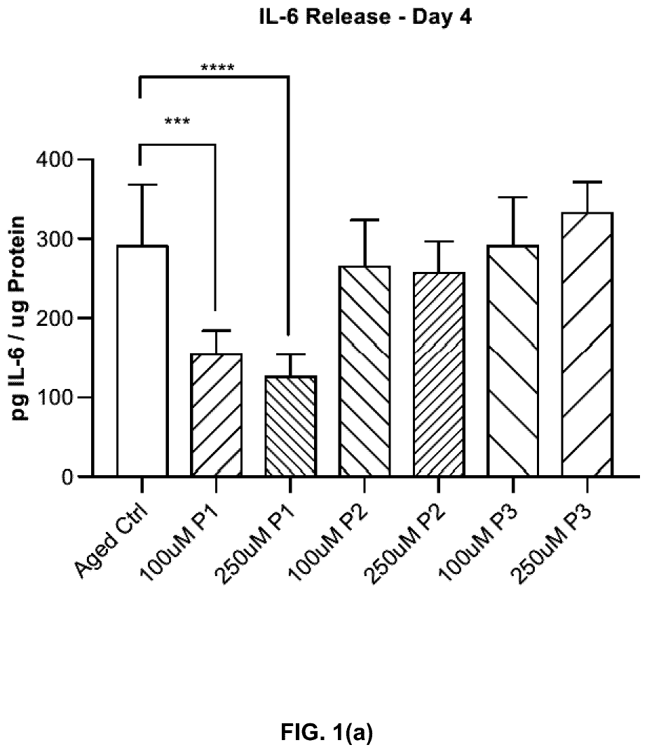

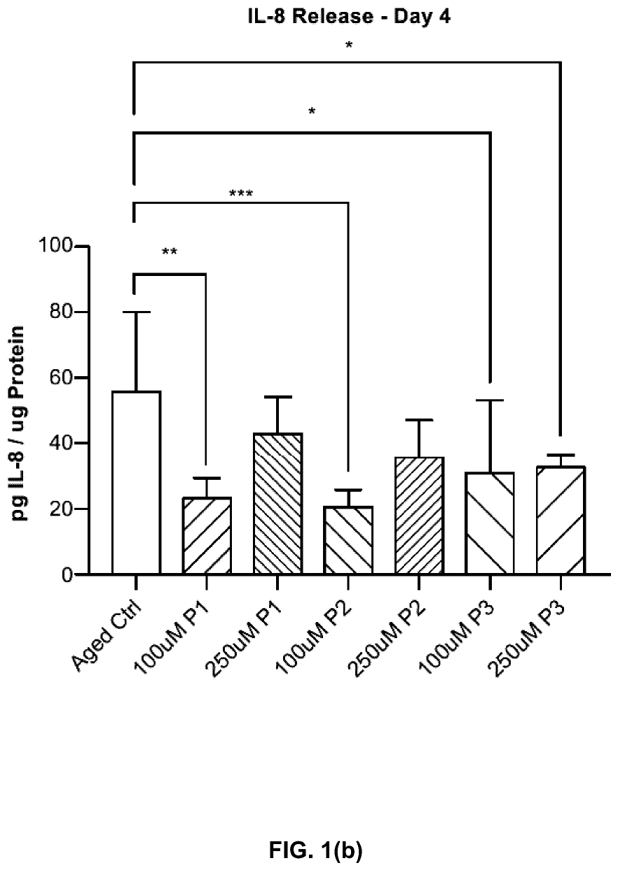

Adult Dermal Human Fibroblasts (Lot 2485, Passage 5, Cell Applications) were expanded and seeded into a 48-well plate at a density of 5,000 cells/well. Cells were grown in Eagle's Minimal Essential Medium (EMEM) supplemented with Fetal Bovine Serum (FBS) and Penicillin-Streptomycin. Cells attached overnight and then culture media was aspirated and replaced with media containing either 100 uM or 250 uM of Peptide I, Peptide II, or Peptide III. Cells were cultured for 4 days with treatment with renewal of treatment medium every other day with the exception of weekends. Media was collected at the end of the culture period. Inflammatory cytokines were quantified by running a LEGENDplex Human Inflammation Panel 1 13-plex (Catalogue #740809, Biolegend. Protein in the media supernatant was also quantified using the Pierce BCA Protein Assay Kit (Catalogue #23225, ThermoFisher). Microsoft Excel and GraphPad Prism were used for additional analysis and data presentation. The amounts of Interleukin 6 (IL-6) and Interleukin 8 (IL-8) released from the Adult Dermal Human Fibroblasts are shown below in Table 1 and graphically depicted in FIG. 1(a) (for IL-6) and FIG. 1(b) (for IL-8).

TABLE 1

| | | Pg IL-6/μg | Pg IL-8/μg |
|---|---|---|---|
| Peptide | Concentration | Protein | Protein |
| Aged Control | | 292.81 ± 76.91 | 55.69 ± 24.27 |
| Peptide I | 154.52 ± 32.33 | 154.52 ± 32.33 | 22.72 ± 6.45 |
| Peptide I | 126.97 ± 29.10 | 126.97 ± 29.10 | 42.71 ± 11.29 |
| Peptide II | 265.86 ± 58.08 | 265.86 ± 58.08 | 20.36 ± 5.39 |
| Peptide II | 257.53 ± 38.82 | 257.53 ± 38.82 | 35.68 ± 11.37 |
| Peptide III | 291.73 ± 61.03 | 291.73 ± 61.03 | 31.14 ± 22.26 |
| Peptide III | 333.61 ± 38.37 | 333.61 ± 38.37 | 32.58 ± 4.06 |

### Example 2

### Increased Growth Factor

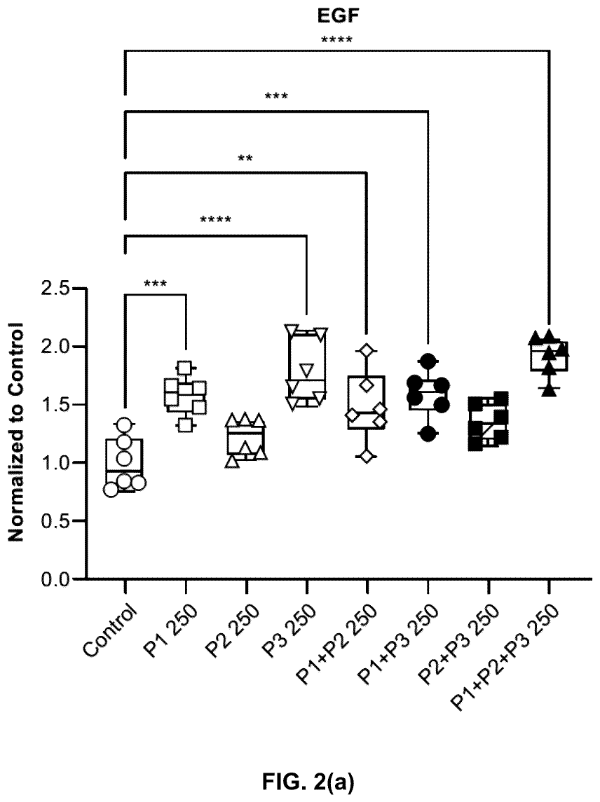

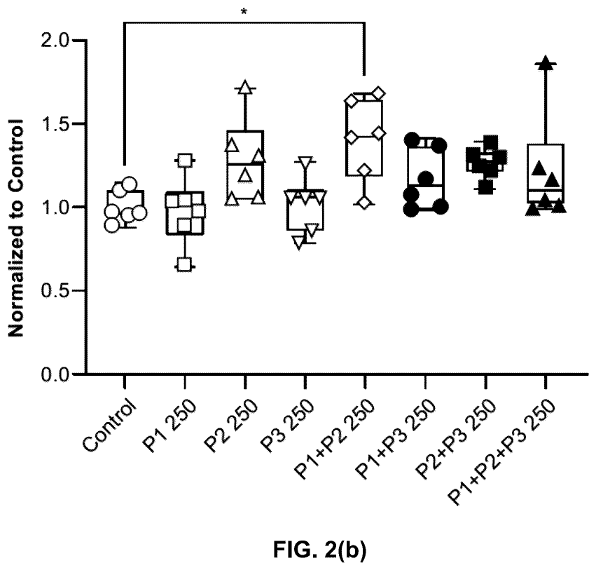

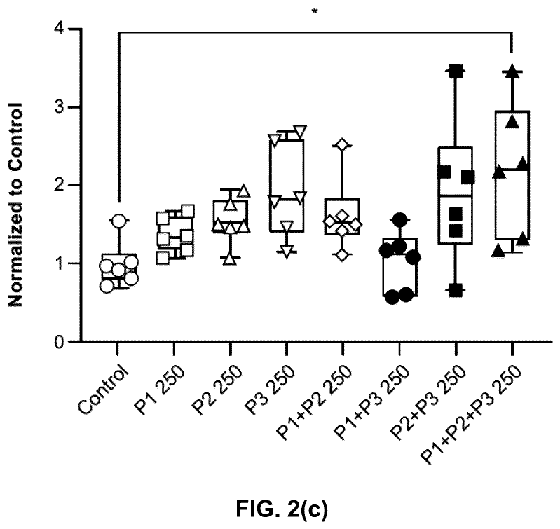

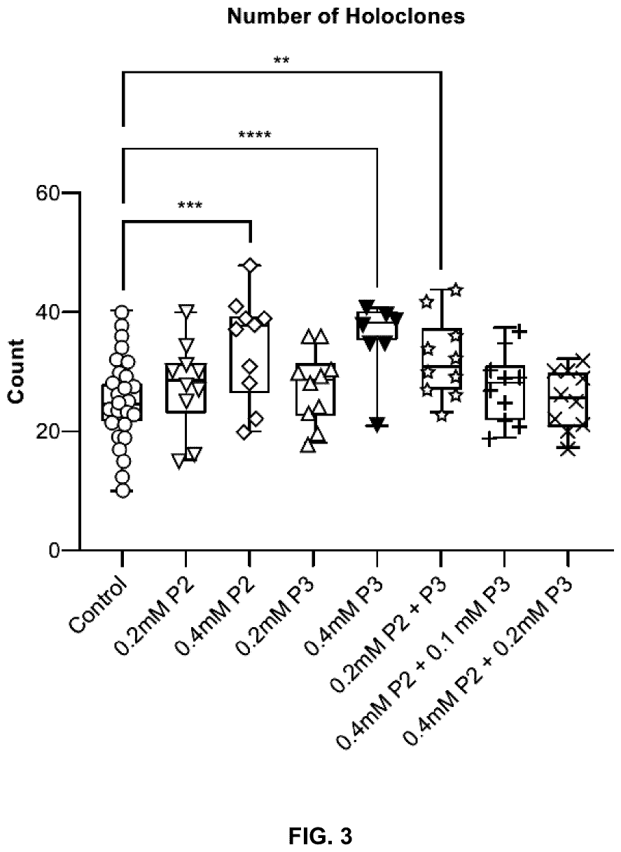

Reconstructed human epidermis (RHE) (EPISKIN/S/13, Episkin) was received and samples were prepared for use. Upon arrival, inserts containing the RHE were removed from the multi-plate and placed in a 12-well plate containing maintenance medium. Tissue serving as untreated control were placed in regular maintenance medium; treatment groups were placed in maintenance medium containing 250 uM of Peptide I, II, or III alone or in combination. Medium was aspirated and replaced every other day with the exception of weekends. RHE was cultured at air-liquid interface at 37C, 5% CO₂ and saturated humidity for 7 days. Media was collected at the end of the culture period. Growth factors were quantified by running a LEGENDplex Human Growth Factor Panel (Catalogue #740809, Biolegend. Protein in the media supernatant was also quantified using the Pierce BCA Protein Assay Kit (Catalogue #741061, ThermoFisher). Microsoft Excel and GraphPad Prism were used for additional analysis and data presentation. The amounts of epidermal growth factor (EGF), hepatocyte growth factor (HGF), and transforming growth factor alpha (TGFa) generated by the RHE are shown below in Table 2 and graphically shown in FIG. 2(a) (for EGF) and FIG. 2(b) (for HGF), and FIG. 2(c) (for TGFa).

TABLE 2

| | Concen- | EGF/μg | HGF/μg | TGFa/μg |
|---|---|---|---|---|
| Peptide | tration | Protein | Protein | Protein |
| Control | | 1.00 ± 0.23 | 1.00 ± 0.09 | 1.00 ± 0.29 |
| Peptide I | 250 μM | 1.58 ± 0.17 | 0.98 ± 0.20 | 1.37 ± 0.23 |
| Peptide II | 250 μM | 1.23 ± 0.16 | 1.28 ± 0.25 | 1.56 ± 0.29 |
| Peptide III | 250 μM | 1.79 ± 0.28 | 1.01 ± 0.17 | 1.93 ± 0.60 |
| Peptides I + II (1:1) | 250 uM | 1.49 ± 0.31 | 1.40 ± 0.25 | 1.62 ± 0.47 |
| Peptides I + III (1:1) | 250 uM | 1.59 ± 0.21 | 1.16 ± 0.18 | 1.04 ± 0.39 |
| Peptides II + III (1:1) | 250 μM | 1.36 ± 0.16 | 1.26 ± 0.09 | 1.92 ± 0.94 |
| Peptide I + II + III | 250 μM | 1.92 ± 0.16 | 1.21 ± 0.33 | 2.21 ± 0.88 |
| (1:1:1) | | | | |

### Example 3

### Increased Epidermal Stem Cells

Keratinocyte stem cells (Lot KSC870, Passage 2, Bioalternatives) were seeded in 96-well plates and cultured in Keratinocyte-SFM supplemented with Epidermal Growth Factor and Pituitary Extract. After 4 hours, media was replaced with that containing Peptide II or Peptide III, alone or in combination. Cells were incubated for 96 hours with treatment renewal after 24 hours. After 96 hours, the medium was removed, and cells were fixed with methanol at −20C. Cells were then stained for 10 minutes with a Papanicolaou solution and imaged. The number of holoclone clones were counted. Data was analyzed and plotted using GraphPad Prism. The holocolone count relative to control (normalized) is shown below in Table 3 for different concentrations of peptide II, Peptide III, and combinations of Peptide II and Peptide III. The results are graphically shown in FIG. 3.

TABLE 3

| | | Holoclone Count to |
|---|---|---|
| | | Control |
| Peptide | Concentration | (normalized) |
| Control | | 24.80 ± 6.14 |
| Peptide II | 0.2 mM | 27.60 ± 7.59 |
| Peptide II | 0.4 mM | 34.30 ± 8.85 |
| Peptide III | 0.2 mM | 27.40 ± 6.13 |
| Peptide III | 0.4 mM | 36.50 ± 5.97 |
| Peptide I + III (1:1) | 0.2 mM | 32.30 ± 6.82 |
| Peptide II + III (4:1) | 0.4 mM Peptide II | 27.40 ± 5.85 |
| | 0.1 mM Peptide III | |
| Peptide II + III (2:1) | 0.4 mM Peptide II | 25.20 ± 5.05 |
| | 0.2 mM Peptide III | |

### Example 4

### Induced Autophagy in Aged Fibroblasts

Adult Dermal Human Fibroblasts (Lot 2485, Passage 5, Cell Applications) were expanded and seeded into a 12-well glass bottom plate at a density of 10,000 cells/well. Cells were grown in Eagle's Minimal Essential Medium (EMEM) supplemented with Fetal Bovine Serum (FBS) and Penicillin-Streptomycin. Cells attached overnight and then culture media was aspirated and replaced with media containing either 100 uM or 250 uM of Peptide I, Peptide II, or Peptide III. Cells were cultured for 7 days with treatment with renewal of treatment medium every other day with the exception of weekends. On day 7, cells were stained and imaged for presence of autophagic vacuoles following the protocol in the Autophagy Assay Kit (ab139484, Abcam). The images were compared to determine whether the number of autophagic vacuoles increased, decreased, or remained unchanged. The results are reported below, in Table 4, where “↑” indicates and increase, “↓” indicates a decrease, and “↔” indicates no change.

TABLE 4

| | | Autophagic Vacuoles Presence |
|---|---|---|
| Peptide | Concentration | (Compared to Control) |
| Control | | n/a |
| Peptide I | 100 μM | ↑ |
| Peptide I | 250 μM | ↑ |
| Peptide II | 100 μM | ↑ |
| Peptide II | 250 μM | ↑ |
| Peptide III | 100 μM | ↑ |
| Peptide III | 250 μM | ↔ |

### Example 5

### Improved Mitochondrial Function in Aged Fibroblasts

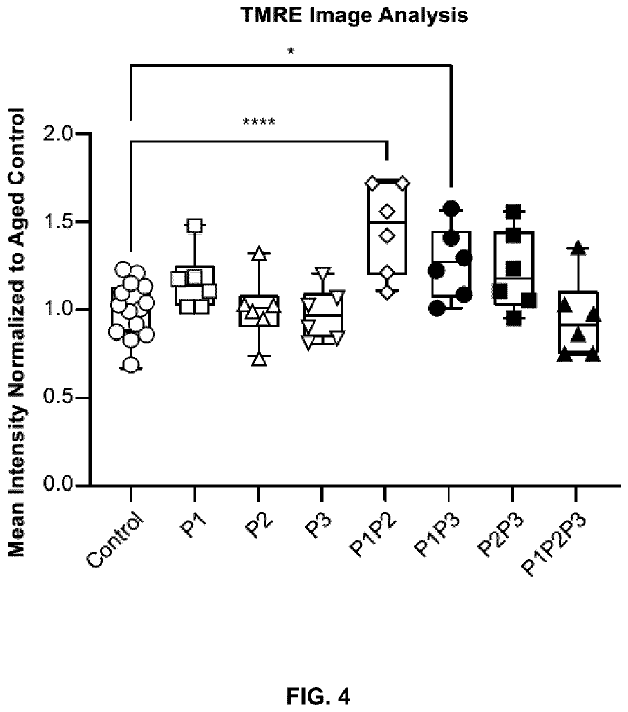

Adult Dermal Human Fibroblasts (Lot 2485, Passage 5, Cell Applications) were expanded and seeded into a 12-well glass bottom plate at a density of 10,000 cells/well. Cells were grown in Eagle's Minimal Essential Medium (EMEM) supplemented with Fetal Bovine Serum (FBS) and Penicillin-Streptomycin. Cells attached overnight and then culture media was aspirated and replaced with media containing either 100 uM or 250 uM of Peptide I, Peptide II, or Peptide III. Cells were cultured for 7 days with treatment with renewal of treatment medium every other day with the exception of weekends. On day 7, cells were stained with tetramethylrhodamine ethyl ester (TMRE) and imaged for mitochondrial activity following the protocol in the TMRE-Mitochondrial Membrane Potential Assay Kit (ab113852, Abcam). The active mitochondria (mean intensity normalized to control) were determined and are shown below, in Table 5. The results are graphically shown in FIG. 4.

TABLE 5

| | | Active Mitochondria |
|---|---|---|
| | | (Mean Intensity |
| Peptide | Concentration | Normalized to Control) |
| Control | | 1.00 ± 0.15 |
| Peptide I | 250 μM | 1.158 ± 0.17 |
| Peptide II | 250 μM | 1.00 ± 0.19 |
| Peptide III | 250 μM | 0.97 ± 0.15 |
| Peptides I + II (1:1) | 250 μM | 1.45 ± 0.26 |
| Peptides I + III (1:1) | 250 μM | 1.26 ± 0.21 |
| Peptides II + III (1:1) | 250 μM | 1.21 ± 0.23 |
| Peptide I + II + III (1:1:1) | 250 μM | 0.95 ± 0.22 |

### Example 6

### Face Treatment

An example face treatment containing about 0.01 to about 1 wt. % of Peptide I, Peptide II, Peptide III, or combinations thereof, is set forth below.

TABLE 6

| INGREDIENTS | Wt. % |
|---|---|
| PEPTIDE I, II, III, OR COMBINATIONS THEREOF | 0.01-2 |
| METHYLPARABEN | 0.3 |
| 2-PHENOXYETHANOL | 0.5 |
| SODIUM POLYACRYLATE | 0.8 |
| XANTHAN GUM | 0.2 |
| WATER/AQUA | 98.2 |

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both an emollient and a nonionic surfactant. If a particular composition includes both an emollient and a nonionic surfactant, a single compound will serve as only the emollient or only as the nonionic surfactant (the single compound does not simultaneously serve as both the emollient and nonionic surfactant).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for treating skin comprising topically applying a therapeutically effective amount of one or more bioactive walnut peptides to the skin.

2. The method of claim 1, wherein the one or more bioactive walnut peptides have a molecular weight of less than 5,000 Da, preferably less than 2,000 Da, more preferably less than 1,000 Da.

3. The method of claim 1, wherein the one or more bioactive walnut peptides have 2 to 20 amino acid residues.

4. The method of claim 1, wherein the one or more bioactive walnut peptides include one or more amino acids selected from leucine, proline, and combinations thereof.

5. The method of claim 1, wherein the one or more bioactive walnut peptides include three or more amino acids selected from leucine, proline, or combinations thereof.

6. The method of claim 1, wherein the one or more bioactive walnut peptides comprise an amino acid sequence selected from Leu-Pro-Leu, Leu-Leu-Pro, Pro-Pro-Leu, or Pro-Leu-Pro.

7. The method of claim 1, wherein the one or more bioactive walnut peptides comprise an amino acid sequence selected from Thr-Trp-Leu-Pro-Leu-Pro-Arg, Tyr-Val-Leu-Leu-Pro-Ser-Pro-Lys, or Lys-Val-Pro-Pro-Leu-Leu-Tyr.

8. The method of claim 1, wherein the one or more bioactive walnut peptides are selected from:
Peptide I
TWLPLPR (Thr-Trp-Leu-Pro-Leu-Pro-Arg),
Peptide II
YVLLPSPK (Tyr-Val-Leu-Leu-Pro-Ser-Pro-Lys),
Peptide III
KVPPLLY (Lys-Val-Pro-Pro-Leu-Leu-Tyr),
or
combination thereof.

9. The method of claim 1, wherein the method comprises topically applying a combination of two or more bioactive walnut peptides to the skin.

10. The method of claim 1, wherein the method comprises topically applying a combination of three or more bioactive walnut peptides to the skin.

11. The method of claim 1, wherein the method:
(a) improves the appearance of skin;
(b) reduces, treats, and/or prevents skin aging and/or the effects of skin aging;
(c) increases fibroblast proliferation;
(d) stimulates epithelial cell proliferation, motility, morphogenesis, and/or angiogenesis;
(e) promotes migration of fibroblasts;
(f) increases abundance or synthesis of hyaluronic acid synthesis, and/or increases synthesis or abundance of collagen, elastin, and/or fibronectin;
(g) improves elasticity of skin;
(h) improving autophagy in skin fibroblasts;
(i) depigments the skin,
(j) lightens the skin,
(k) brightens the skin,
(l) treats hyperpigmentation, and/or
(m) treats melasmic skin, and/or
(n) evens out skin tone.

12. The method of claim 1, wherein the method reduces, treats, or prevents production, release, or abundance of pro-inflammatory cytokines in the skin.

13. The method of claim 1, wherein the method potentiates production, release, or abundance of epidermal growth factor (EGF), hepatocyte growth factor (HGF), transforming growth factor alpha (TGFa), or combinations thereof.

14. The method of claim 1, wherein the method potentiates, induces, and/or increases autophagy in skin.

15. The method of claim 1, wherein the method improves mitochondrial function and/or activity of the skin.

16. The method of claim 1, wherein the method comprises topically applying a combination of two or more bioactive walnut peptides to the skin, wherein topical application of the combination of the two or more walnut peptides provides a synergistic effect that is greater than the additive sum provided by topical application of corresponding amounts of the individual bioactive walnut peptides.

16. A therapeutic or cosmetic composition for topical application to the skin comprising the one or more walnut peptides of claim 1.

17. The composition of claim 16, wherein the two or more walnut peptides include a first bioactive walnut peptide and a second bioactive walnut peptide, and the first bioactive walnut peptide and the second bioactive walnut peptide are in a weight ratio of about 1:10 to about 10:1.

18. The composition of claim 17, comprising three or more bioactive walnut peptides.

19. The composition of claim 18, wherein the three or more bioactive walnut peptides include a first bioactive walnut peptide, a second bioactive walnut peptide, and a third bioactive walnut peptide, wherein:
(i) the first bioactive walnut peptide and the second bioactive walnut peptide are in a weight ratio of about 1:10 to about 10:1;
(ii) the first bioactive walnut peptide and the third bioactive walnut peptide are in a weight ratio of about 1:10 to about 10:1; and
(iii) the second bioactive walnut peptide and the third bioactive walnut peptide are in a weight ratio of about 1:10 to about 10:1.

20. The composition of claim 16 comprises about 0.001 to about 10 wt. % of the one or more bioactive walnut peptides, and a physiologically acceptable carrier.
